(19) United States
(12) Patent Application Publication
(10) Pub. No.: US 2008/0227183 A1
(43) Pub. Date: Sep. 18, 2008

(54) APPARATUS FOR SUPPORTING DIAGNOSIS OF CANCER

(76) Inventors: Hideki Ishihara, Miki-shi (JP); Yasuhiro Kouchi, Kakogawa-shi (JP)

Correspondence Address: BRINKS HOFER GILSON & LIONE, P.O. BOX 10395, CHICAGO, IL, 60610, US

(21) Appl. No.: 12/048,103

(22) Filed: Mar. 13, 2008

(30) Foreign Application Priority Data
Mar. 14, 2007 (JP) 2007-065029

(51) Int. Cl. C12M 1/34; G01N 33/48

(52) U.S. Cl. 435/287.1; 702/19

(57) ABSTRACT

A cancer diagnosis supporting apparatus is provided with a memory for storing a predetermined reference value, a diagnosis support information preparer for preparing the diagnosis support information of cancer by comparing a measurement value obtained from a malignant tumor collected from a cancer patient and the reference value, and a change acceptor for accepting change of the reference value and for storing the changed reference value in the memory.

### RELATED APPLICATIONS

This application claims priority under 35 U.S.C. § 119 to Japanese Patent Application Nos. JP2007-065029 filed Mar. 14, 2007, the entire contents of which are hereby incorporated by reference.

### FIELD OF THE INVENTION

The present invention relates to an apparatus for supporting a diagnosis of cancers.

### BACKGROUND

Serum diagnosis for examining the tumor marker in the serum, as well as tissue diagnosis and cell diagnosis by biopsy are conventionally known for cancer diagnosis. However, since the reliability thereof is low or the determination of the individual or determination of medical facilities varies, a molecular diagnosis based on genes and protein expressed in the living body is recently reviewed as a standardized diagnosis method of cancer in which variation among diagnostician is small. Various methods such as a method using cyclin-dependent kinase (hereinafter simply referred to as “CDK”) have been proposed as a molecular diagnosis based on protein.

US 2003-134315 discloses a diagnosis method having an expression level of CDK1 and CDK4 of a sample, and furthermore, optionally mutation state of p53 as index. US 2003-152993 discloses a diagnosis method of cancerous and precancerous states having excessive expression of CDK4, CDK6, and cyclin-dependent kinase inhibitor (CDK inhibitor) as index. US 2002-164673 discloses a method of measuring an activity value of the CDK using fluorescence and a method of diagnosing cancer using the same. US 2007-231837 discloses a method of determining malignancy of cancer with the ratio of the activity value of CDK1 and CKD2 and the expression level as the index.

When making the determination using a specific measurement value such as expression level and specific activity of the CDK as the index, a specific reference value is normally set as the index, and various determinations are made based on the comparison between such reference value and the measurement value obtained by measuring the sample collected from the patient.

In diagnosing cancer, information on the measurement value related to the index, presence of lymph node metastasis, content of postoperative therapy (no therapy, hormonal therapy, chemotherapy, etc.), presence of recurrence, and clinical information such as number of days from when cancer is extirpated until recurrence are acquired from a great number of patients, the information (sample data) are accumulated as library information, and a value at which determination can be made at best accuracy based on the library information is set as the reference value.

That is, the reference value of the index is not necessarily defined or fixed, and the reference value sometimes needs to be changed when the library information is updated or added.

In view of the above, it is an object of the present invention to provide a diagnosis support apparatus of cancer in which the reference value of the index used in diagnosing cancer can be changed by the user.

### BRIEF SUMMARY

The scope of the present invention is defined solely by the appended claims, and is not affected to any degree by the statements within this summary. A cancer diagnosis supporting apparatus embodying features of the present invention includes: a memory for storing a predetermined reference value; a measurement value acquirer for acquiring a measurement value of a predetermined item from a malignant tumor collected from a cancer patient; a diagnosis support information preparer for preparing the diagnosis support information of cancer by comparing the acquired measurement value and the reference value; and a change acceptor for accepting change of the reference value and for storing the changed reference value in the memory.

### BRIEF DESCRIPTION OF THE DRAWINGS

FIG. 1 is a perspective explanatory view of one embodiment of a cancer diagnosis supporting apparatus of the present invention.

FIG. 2 is a perspective explanatory view of a chip setting unit and a solid phase tip for protein in the cancer diagnosis supporting apparatus shown in FIG. 1.

FIG. 3 is a cross sectional explanatory view of the chip setting unit and the solid phase tip for protein in the cancer diagnosis supporting apparatus shown in FIG. 1.

FIG. 4 is an exploded explanatory view of an upper plate and a lower plate of the solid phase tip for protein.

FIG. 5 is a perspective explanatory view of the solid phase tip for protein with the upper plate attached to the lower plate.

FIG. 6 is a cross sectional explanatory view of a column of a sample preparation unit of the activity measurement unit in the cancer diagnosis supporting apparatus shown in FIG. 1.

FIG. 7 is a perspective view of the sample preparation unit of the activity measurement unit in the cancer diagnosis supporting apparatus shown in FIG. 1.

FIG. 8 is a top view of a fluid manifold of the sample preparation unit shown in FIG. 7.

FIG. 9 is a cross sectional view taken along line D-D of FIG. 8.

FIG. 10 is a fluid circuit diagram of the sample preparation unit shown in FIG. 7.

FIG. 11 is a block diagram showing a control system for controlling the cancer diagnosis supporting apparatus.

FIG. 12 is a block diagram showing a hardware configuration of a data processing unit.

FIG. 13 is a block diagram showing a hardware configuration of a main body controller.

FIG. 14 is a view describing a cell cycle;

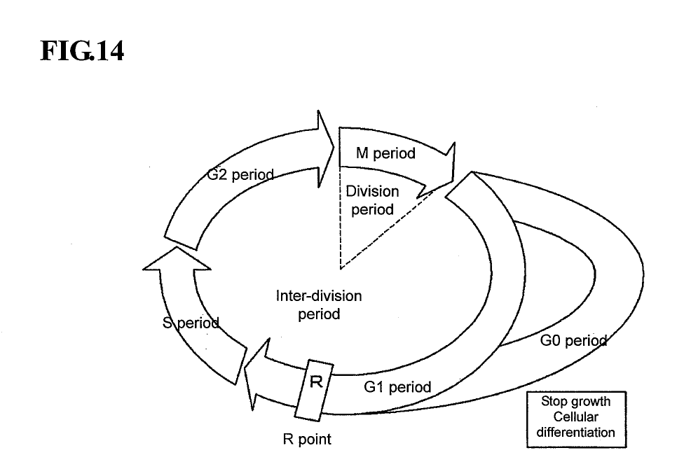

FIG. 15 is a flowchart showing one example of a process by the cancer diagnosis supporting apparatus.

FIG. 16 is a flowchart showing one example of a process by the cancer diagnosis supporting apparatus.

FIG. 17 is flowchart showing one example of a process by the cancer diagnosis supporting apparatus.

FIG. 18 is a flowchart showing a preparation process of the expression level measurement sample.

FIG. 19 is a flowchart showing a preparation process of the activity value measurement sample.

FIG. 20 is an explanatory view showing usage procedures of specimen etc. in the cancer diagnosis supporting apparatus.

FIG. 21 is a flowchart showing one example of an analyzing process in the cancer diagnosis supporting apparatus.

FIG. 22 is a view showing an example of a diagnosis support information display screen.

FIG. 23 is a view showing an example of a reference value changing screen (before change).

FIG. 24 is a view showing an example of a reference value changing screen (after change).

FIG. 25 is a view showing an example of sample data.

FIG. 26 is a view showing an example of sample data.

FIG. 27 is a flowchart showing another example of a process by the cancer diagnosis supporting apparatus.

FIG. 28 is a flowchart showing another example of a process by the cancer diagnosis supporting apparatus.

FIG. 29 is a flowchart showing another example of a process by the cancer diagnosis supporting apparatus.

FIG. 30 is a flowchart showing another example of an analyzing process by the cancer diagnosis supporting apparatus.

FIG. 31 is a flowchart showing another further example of an analyzing process by the cancer diagnosis supporting apparatus.

FIG. 32 is a view showing an example of a reference value changing screen in performing determination of three stages.

FIG. 33 is a view showing an example of a reference value changing screen using HER2 as an index other than CDK1 and CDK2.

FIG. 34 is a view showing an example of a reference value changing screen using CDK2 and p21 as indices.

### DETAILED DESCRIPTION OF THE PREFERRED EMBODIMENTS

An embodiment of a cancer diagnosis supporting apparatus (hereinafter also referred to simply as “diagnosis support apparatus”) of the present invention will now be described in detail with reference to the accompanying drawings.

The diagnosis support apparatus of the present embodiment measures predetermined items using malignant tumor collected from a cancer patient, and acquires diagnosis support information of cancer based on the obtained measurement value.

Malignant tumors are tumors that invade or metastasize to other tissues, and enlarge at various sites of the body thereby threatening the human life. The malignant tumor includes cancer or malignant tumor originating from epithelial tissue, and sarcoma or malignant tumor originating from non-epithelial tissue. Specifically, the malignant tumor includes malignant tumors forming at positions such as breast, lung, liver, stomach, large intestine, pancreas, uterus, testis, ovaria, thyroid, accessory thyroid, lymphography, and the like. The malignant tumor can be collected from cancer patients having breast cancer, lung cancer, liver cancer, gastric cancer, large intestine cancer, pancreas cancer, prostate cancer, and the like.

The diagnosis support information of cancer includes malignancy of cancer, receptivity to anticancer drug, and the like. The malignancy of cancer specifically includes likelihood to metastasize, likelihood to recurrence, unsatisfactory prognosis, and the like.

The predetermined item measured in the diagnosis support apparatus is not particularly limited as long as it is a measurement value related to gene and/or protein in the malignant tumor, and the type of gene or protein and the type of measurement item are appropriately selected depending on the type of cancer, the diagnosis support information to be acquired, or the like.

The predetermined item includes measurement value related to CDK disclosed in US2003-134315, US2003-152993, US2002-164673, and US2007-231837. Specifically, the predetermined item includes expression level of CDK, activity value of CDK, ratio (specific activity ratio) of the activity value and the expression level of the CDK, and the like. In addition to the CDK, the predetermined item may be the expression level of the gene used to predict the recurrence risk of the cancer in JP 2005-58113 and US2007-264635. Furthermore, the predetermined item may be the expression level of the gene used to predict the receptivity of anticancer drug in the International Publication No. 2005/007846 pamphlet, Japanese Laid-Open Patent Publication No. 2005-341862, and the like.

The diagnosis support apparatus of the present embodiment will now be described using a diagnosis support apparatus for measuring the expression level and the activity value of the CDK using the malignant tumor collected from the cancer patient, and determining the malignancy of the cancer (possibility of recurrence risk) based on the obtained measurement value, by way of example.

Prior to describing the diagnosis support apparatus, [1] a method of determining the malignancy of the cancer using the CDK will be described.

[1] Method of Determining the Malignancy of Cancer Using CDK

The method of determining malignancy of cancer includes measuring the expression level and the activity value of two or more types of cyclin-dependent kinases of a tissue containing the malignant tumor, and determining the malignancy of the tumor tissue based on the CDK profile including the ratio of the activity value and the expression level of a first cyclin-dependent kinase, and a ratio of the activity value and the expression level of a second cyclin-dependent kinase. The character of the tissue containing the tumor cell and the malignancy of the cancer can be diagnosed by applying the determining method on the tissue containing the tumor cell. The CDK profile is information including ratio (e.g., specific activity) of the activity value and the expression level of at least one type of CDK of a certain tissue and/or numerical value calculated with the activity value and the expression level of a plurality of CDKs (e.g., ratio of the ratio (A1) of the activity value and the expression level of the first CDK, and the ratio (A2) of the activity value and the expression level of the second CDK (e.g., A1/A2 or A2/A1).

The malignant tumor described above can be used in the determining method. The malignancy of cancer specifically includes likelihood to metastasize, likelihood to recurrence, unsatisfactory prognosis, and the like.

Here, recurrence refers to a case where the same malignant tumor reappears in the remaining organs after an organ is partially removed to extirpate the malignant tumor, and a case where the tumor cell is separated from a primary tumor and conveyed to a remote tissue (remote organ), and independently grows thereat (metastasize and recur). Generally, “likely to recur” refers to a case where there is a possibility of recurring in five or less years. This is because the death rate of the patients recognized with recurrence in five or less years is high, and thus, predicting the recurrence of five or less years after the extirpative operation has clinical meaning. In stage classification, stage III has a rate of recurrence of 50%, and recurrence is likely to occur compared to stage II (rate of recurrence of 20%). Prognosis means anticipating the elapse and the end of illness, where prognosis is poor the higher the death rate after five years or ten years, and for example, stage III has death rate of 50% and thus has poorer prognosis than stage II (death rate 20%).

The cyclin-dependent kinase is a collective term of enzyme group activated by being bounded to cyclin, and functions in a specific time of the cell cycle depending on the type thereof. The CDK inhibitor is a collective term of a factor group that bonds with the cyclin CDK complex and inhibits the activity thereof.

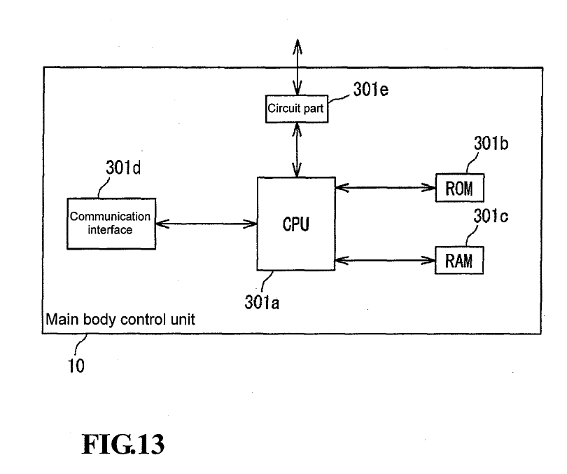

The cell cycle which is a cycle of when the cell starts growing and returns to the starting point as two daughter cells after events of DNA replication, distribution of chromosomes, nuclear division, cytoplasmic division, and the like is divided into four periods of G1 period, S period, G2 period, and M period, as shown in FIG. 13. The S period is the replication period of the DNA, and the M period is the division period. The G1 period is a preparation period for entering the M period between the completion of mitotic division and the start of DNA synthesis. After passing a critical point (point R in animal cell) in the G1 period, the cell cycle starts, and normally completes one cycle without stopping in the middle. The G2 period is between the termination of the DNA synthesis and the start of mitotic division. Main check points of the cell cycle is immediately before entering the S period from the G1 period, and the entrance to mitotic division from the G2 period. In particular, the G1 period check point is important because it triggers the start of the S period. After passing a certain point of the G1 period, the cell advances the cell cycle as S→G2→M→G1 without stopping the growth even if a growth signal is not provided. A rest period (G0) having DNA content of the G1 period is created in the cell that has stopped growing, which is in a state deviated from the cell cycle. Due to growth induction, advancement to the S period is made after a time slightly longer than the G1 period in the cell cycle.

The cyclin-dependent kinase (CDK) used in the determining method is preferably selected from a group consisting of CDK1, CDK2, CDK4, CDK6, cyclin A-dependent kinase, cyclin B-dependent kinase, cyclin D-dependent kinase, and cyclin E-dependent kinase. The cyclin A-dependent kinase is the CDK that indicates activity by being bound to cyclin A, and it is currently known to refer to both CDK1 and CDK2. The cyclin B-dependent kinase is the CDK that indicates activity by being bound to cyclin B, and it is currently known to refer to CDK1. The cyclin D-dependent kinase is the CDK that indicates activity by being bound to cyclin D, and it is currently known to refer to both CDK4 and CDK6. The cyclin E-dependent kinase is the CDK that indicates activity by being bound to cyclin E, and it is currently known to refer to CDK2.

It is currently known that such CDK activates a specific period of the cell cycle as shown in table 1 by being a cyclin-CDK complex (hereinafter also referred to as “active CDK”) bound to the corresponding cyclin, as shown in table 1. For instance, CDK1 becomes active by binding to cyclin A or B, CDK2 becomes active by binding to cyclin A or E, and CDK4 and CDK6 become active by binding to cyclin D1, D2, or D3. The CDK activity sometimes has the activity inhibited by the CDK inhibitor as shown in table 1. For instance, p21 inhibits CDK1 and CDK2, p27 inhibits CDK2, CDK4, and CDK6, and p16 inhibits CDK4 and CDK6.

TABLE 1

| | | INTEGRATING CDK | OPERATING PERIOD OF |
|---|---|---|---|
| CDK | INTEGRATING CYCLIN | INHIBITOR | ACTIVE CDK |
| CDK4 | CYCLIN D1 | p27, p16 | G1 |
| CDK6 | CYCLIN D2 | | |
| | CYCLIN D3 | | |
| CDK2 | CYCLIN E | p27 | G1 → S TRANSITION |
| CDK2 | CYCLIN A | p21, p27 | S PERIOD ACTIVE |
| CDK1 | CYCLIN A, | p21 | G1 → M TRANSITION |
| | CYCLIN B | | |
| CYCLIN A-DEPENDENT KINASE | CYCLIN A | p21, p27 | CDK1: G1 → M |
| | | | CDK2: MIDDLE PERIOD |
| | | | OF S PERIOD |
| CYCLIN B-DEPENDENT KINASE | CYCLIN B | p21 | CDK1: G2 → M |
| CYCLIN D-DEPENDENT KINASE | CYCLIN D | p27, p16 | CDK4, 6: G1 |

Among the CDKs, the expression level and the activity level of two or more types of CDKs are measured, the ratio thereof (that is, CDK specific activity or inverse number thereof expressed with the equation below) in each CDK is obtained, and the CDK profile is obtained.

Specific activity of CDK=CDK activity value/CDK expression level

Therefore, the CDK profile specifically includes profile (CDK specific activity profile) containing the CDK specific activity, and profile (inverse number profile of CDK specific activity) containing the inverse number of the CDK specific activity.

The CDK activity value refers to the level (unit is expressed as U (unit)) of the kinase activity on binding with a specific cyclin, and how much substrate (e.g., histon H1 for active CDK1 and active CDK2, and Rb (retinoblastoma protein) for active CDK4 and active CDK6) phosphorylate, and can be measured with a conventionally known enzyme activity measurement method. Specifically, a method of preparing a sample containing the active CDK from the cell dissolved solution of the measurement sample, retrieving ³²P into the substrate protein using ³²P labeled ATP (γ-[³²P]-ATP), measuring the labeled quantity of labeled phosphorylated substrate, and determining the quantity based on the standard curve created with a standard product may be adopted. A method that does not use label of the radioactive substance includes the method disclosed in Japanese Laid-Open Patent Publication No. 2002-335997. This method is a method of preparing a sample containing the target active CDK from the cell solubilizing solution of the measurement sample, reacting adenosine 5′-O-(3-thiotoriphosphate) (ATP-γS) and the substrate, introducing monothiophosphate group into serine or threonine residue of the substrate protein, bonding labeled fluorescence substance or labeled enzyme to the sulfur atom of the introduced monothiophosphate group to label the substrate protein, measuring the labeled quantity (fluorescence quantity when labeled fluorescence substance is used) of the labeled thiophosphate substrate, and determining the quantity based on the standard curve created with the standard product.

The sample provided for activity measurement is prepared by uniquely collecting the target CDK from the solubilizing solution of the tissue containing the malignant tumor to be measured. In this case, the sample may be prepared using a specific anti-CDK antibody for the target CDK, or in the case of activity measurement of certain cyclin-dependent kinase (e.g., cyclin A-dependent kinase, cyclin B-dependent kinase, cyclin E-dependent kinase), the sample may be prepared using an anti-cyclin antibody. In either case, CDK other than the active CDK is contained in the sample. For instance, a complex bound in which the CKD inhibitor is bound to the cyclin CDK complex is also included. When the anti-CDK antibody is used, CDK single body, complex of CDK and cyclin and/or CDK inhibitor, complex of CDK and another compound, and the like are contained. Therefore, the activity value is measured as a unit (U) of phosphorylated substrate under a state that active, non-active, and various competitive reaction coexist.

The CDK expression level is the target CDK level (unit corresponding to number of molecules) measured from the cell solubilizing solution, and is measured with a conventionally known method of measuring the target protein quantity from the protein mixture. For instance, ELISA method, western blot method, and the like may be used, or measurement may be carried out with a method disclosed in Japanese Laid-Open Patent Publication No. 2003-130871. The target protein (CDK) is captured using a specific antibody. For instance, all the CDK1 existing within the cell (include CDK single body, complex of CDK and cyclin and/or CDK inhibitor, complex of CDK and another compound) can be captured using the anti-CDK1 antibody.

Therefore, the specific activity calculated from the above equation corresponds to the proportion of CDK indicating activity of the CDK existing in the cell, and is the CDK activity level based on the growth state of the malignant tumor cell, which is the target of determination. The CDK specific activity obtained in this manner does not depend on the measurement sample preparation method. In particular, the measurement sample (cell solubilizing solution) prepared from the biopsy material is likely to be influenced by the size of non-cellular tissues such as extracellular matrix contained in the actually collected tissue. Therefore, there is a large meaning to using the specific activity or the inverse number thereof in which such influence is eliminated, and the correlation with the clinical characteristics is high compared to the simple activity value of the prior art.

When the CDK profile including the CDK specific activity or the inverse number thereof of two or more types is known, which CDK activity is superior can be known, whereby the extent of the cell proportion in the periods of the cell cycle can be known, or the cell proportion of which period is superior can be known.

The type of CDK for measuring the specific activity is not particularly limited, and may be appropriately selected. Generally, since the cancer cells actively grow deviating from the normal growth control, cell proportion in the S period and the G2 period is assumed to be large, in which case, assumption is made that the cell is becoming cancerous. Such cancer progresses early and is malignant. Furthermore, aneuploid medium is assumed to occur when an abnormal M period has elapsed or enters the G1 period without going through the M period and entering the S period, and thus the cell is assumed to be malignant when the cell proportion in the M period is small. Therefore, the CDK1 is used as the first cyclin-dependent kinase and the CDK2 is used as the second cyclin-dependent kinase, classification to groups is carried out according to the magnitude of the CDK1 specific activity, and the CDK2 specific activity value takes a value reflecting the cell ratio of the S period of the groups having a similar CDK1 specific activity. When there are a great number of cells in the S period, the tissue where the cells are configuring cells is determined as clinically malignant, that is, as a malignant cancer that is likely to metastasize and have poor prognosis.

From the type of CDK and known operation, the existing proportion of a specific period of the cell cycle is estimated from the CDK specific activity profile containing two or more types of CDK specific activities and the malignancy of the cell is determined, or the specific activity profile of two or more types of CDKs where the corresponding normal tissue cell is measured in advance as standard cell is obtained and the malignancy is determined from comparison with the normal cell. The CDK specific activity profile preferably adopts a ratio of specific activity of the two types of cyclin-dependent kinases. In this case, the malignancy is determined by comparing the ratio of the specific activity of the two types of cyclin-dependent kinases with a predetermined reference value or a threshold value corresponding to the ratio.

The reference value used in the determination method is appropriately determined from the type etc. of the malignant tumor to be measured. The setting of the reference value may be carried out by selecting a value of the ratio of the specific activity that acts as a border with respect to malignancy from a database of a great number of cells and individuals related to the malignancy of the cancer and a database on the CDK specific activity of the relevant cell. For instance, regarding the tumor cell collected from a plurality of patients whose judgment of pathologists is known on the malignancy of the cancer, the ratio of the specific activity of two types of CDKs assumed to have correlation is obtained, the obtained ratio is lined in ascending order, and a median value that divides the group in half is set as a reference value.

[2] Diagnosis Support Apparatus

The diagnosis support apparatus according to one embodiment of the present invention in which a method of determining the malignancy of the cancer using [1] CDK described above is suitably performed will be described.

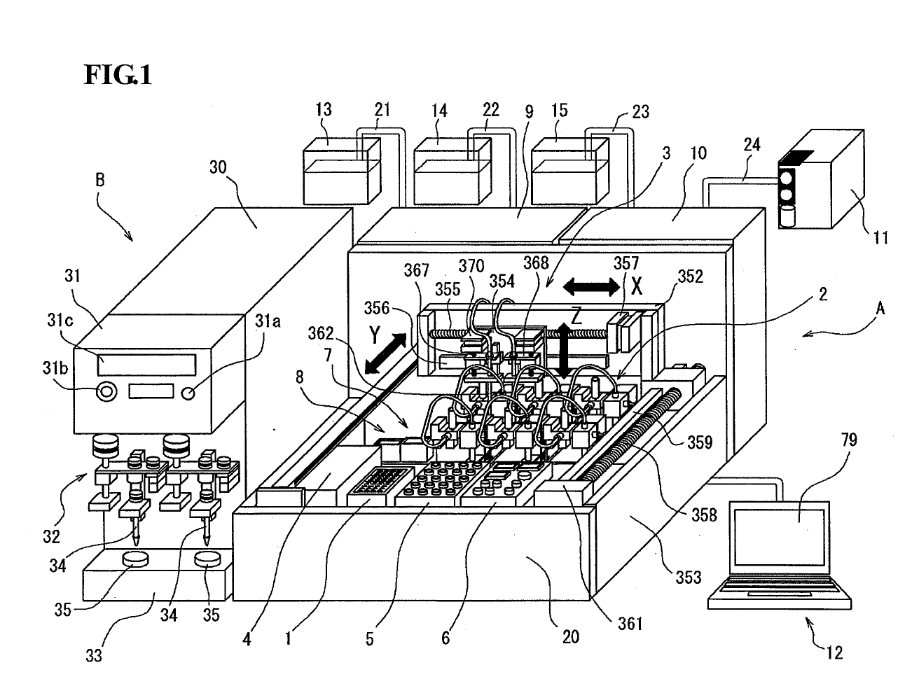

FIG. 1 is a perspective explanatory view of a diagnosis support apparatus A according to one embodiment of the present invention. The diagnosis support apparatus A measures the activity value and the expression level of the protein (cyclin-dependent kinase (CDK) contained in the tissue in the present embodiment), and is mainly configured by a detecting unit 4 arranged at the front portion of an apparatus body 20; a chip setting unit 1; first reagent setting unit 5 and second reagent setting unit 6; an activity measurement unit 2 arranged at a back portion of the apparatus body 20; a waste bath 7 for accommodating waste liquid and a pipette washing bath 8 for washing pipette; a dispensing mechanical member 3 arranged on the upper side of the apparatus body 20, for moving the pipette in three directions (X direction, Y direction, and Z direction); a fluid unit 9 and a main body controller 10 arranged at the back part of the apparatus body 20; and a personal computer 21 which is a data processing unit communicably connected to the main body controller 10. A pure water tank 13, a washing liquid tank 14, a waste tank 15, and a pneumatic source 11 are arranged in the diagnosis support apparatus A of the present embodiment. The pure water tank 13 stores pure water for washing a flow channel at the end of measurement and is connected to the fluid unit 9 via a conduit 21; the washing liquid tank 14 stores washing liquid for washing the pipette and is connected to the pipette washing bath 8 via a conduit 22; and the waste tank 15 for accommodating the waste liquid is connected to the waste bath 7 via a conduit 23. A solubilizing device B for obtaining a specimen that can be processed in the diagnosis support apparatus A from a biological sample is arranged next to the diagnosis support apparatus A.

The solubilizing device B and the diagnosis support apparatus A will be described below in order.

[Solubilizing Device]

Prior to the process by the diagnosis support apparatus A, the solubilizing device B prepares a liquid specimen that can be processed in the diagnosis support apparatus A from the biological sample of the tissue etc. extirpated from the patient, and is mainly configured by a housing 30, an operating member 31 arranged on the upper side at the front surface of the housing 30, a driving member 32 including a pair of pestles 34 for pressing and grinding the biological sample, and a specimen setting member 33 set with an eppen tube 35 accommodating the biological sample.

The driving member 32 moves the pestels 34 in the up and down direction and provides rotational movement thereto, so that the biological sample injected into the eppen tube 35 is pressed and grinded. A controller (not shown) for controlling the operation of the driving member 32 is arranged in the housing 30.

An operation button 31a, an operation light 31b, and a display 31c for displaying the state of the apparatus and error message are arranged on the operating member 31. A cooling means (not shown) is arranged in the sample setting member 33 to maintain the biological sample in the eppen tube set in the concave area of the upper surface of the sample setting member 33 at a constant temperature.

The supernatant solution of the living body reagent solubilized by the solubilizing device B and subjected to centrifugal process by a centrifugal machine (not shown) is extracted to a predetermined sample container and set in the first reagent setting unit 5 of the diagnosis support apparatus A.

[First Reagent Setting Unit]

A cooling means (not shown) is arranged in the first reagent setting unit 5, similar to the sample setting member 33, to maintain the specimen, various antigens such as CDK1 antigen (calibration 1) and CDK2 antigen (calibration 2), and various fluorescent labeled antibodies such as fluorescent labeled CDK1 antibody and fluorescent labeled CDK2 in the container such as screw cap set in the concave area of the upper surface of the first reagent setting unit 5 at a constant temperature. In the present embodiment, a total of 20 concave areas are formed in a matrix of five by four, so that a maximum of 20 containers such as screw cap can be set.

[Second Reagent Setting Unit]

The second reagent setting unit 6 is arranged next to the first reagent setting unit 5. A plurality of concave areas is formed in the second reagent setting unit 6, similar to the first reagent setting unit 5, where containers such as eppen tube and screw cap with buffer, substrate solution, fluorescent enhancement reagent, and the like are set in the concave areas.

Prior to the process by the diagnosis support apparatus A, the solid phase tip for protein is set in the chip setting unit 1, and the column is set in the activity measurement unit 2.

[Chip Setting Unit]

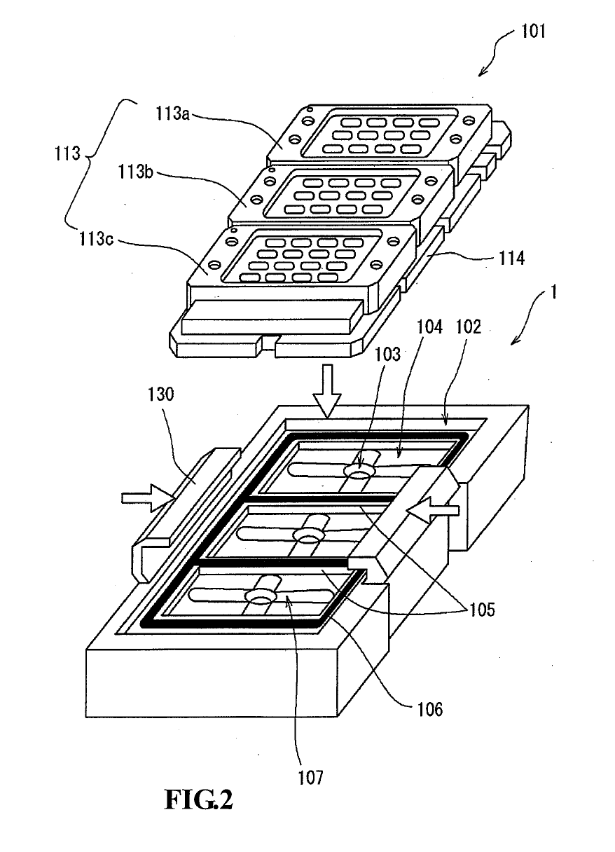

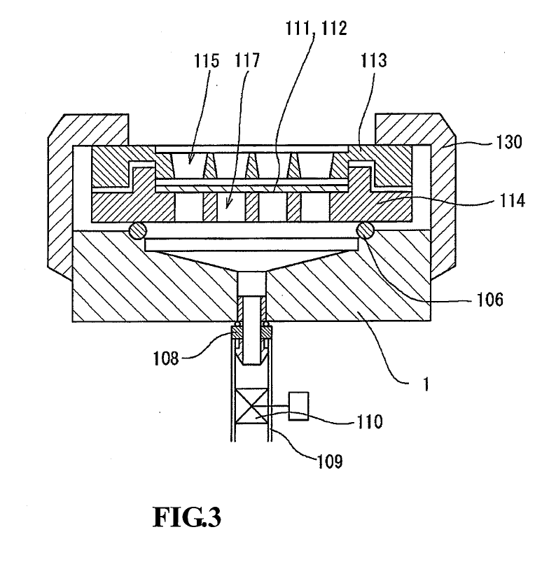

The chip setting unit 1 is made up of aluminum blocks, where a concave part 102 for mounting the solid phase tip for protein 101 is formed at the upper surface and three aspiration ports 103 are formed at the bottom part, as shown in FIGS. 2 and 3. More specifically, the chip setting unit 1 includes a first concave part 102 of rectangular shape at the upper surface, and three second concave parts 104 of rectangular shape at the bottom of the first concave part 102. The second concave parts 104 are independent from each other by a partition wall 105 so as to be in a non-communicating state when the solid phase tip for protein 101 is mounted on the chip setting unit 1. A rubber elastic gasket 106 of rectangular frame shape is arranged on the peripheral edge of the second concave part 104 at the bottom surface of the first concave part 102.

The second concave part 104 includes a cross-shaped groove 107 at the bottom part and the aspiration port 103 at the center of the bottom part, wherein the bottom of the groove 107 is inclined so as to become deeper towards the center from the peripheral edge of the second concave part 104. The aspiration port 103 communicates with a nipple 108 arranged to connect to an external aspiration pneumatic source 11. A tube 109 having one end connected to the aspiration pneumatic source 11 side has the other end connected to the nipple 108. An open/close valve 110 is arranged in the tube 109.

The solid phase tip for protein 101 described below in detail is mounted horizontally at the bottom surface of the first concave part 102 by way of a gasket 106. The aspiration pump is operated after the protein containing sample solution is injected or dropped into each well of the solid phase tip for protein 101.

The solid phase tip for protein 101 is then air tightly attracted to the bottom surface of the first concave part 102 by way of the gasket 106, and the sample solution in each well is aspirated through the porous film described below, whereby the protein to be measured is solid phase formed on the porous film. In FIGS. 2 and 3, 130 is a pressing mechanism for pressing and fixing the solid phase tip for protein 101 to the bottom surface of the first concave part 102. The pressing mechanism 130 is sled in a direction of the arrow in the figure after the solid phase tip for protein 101 is mounted on the first concave part 102, so that the upper part thereof presses the upper surface of the solid phase tip for protein 101 and fixes the same to the first concave part 102.

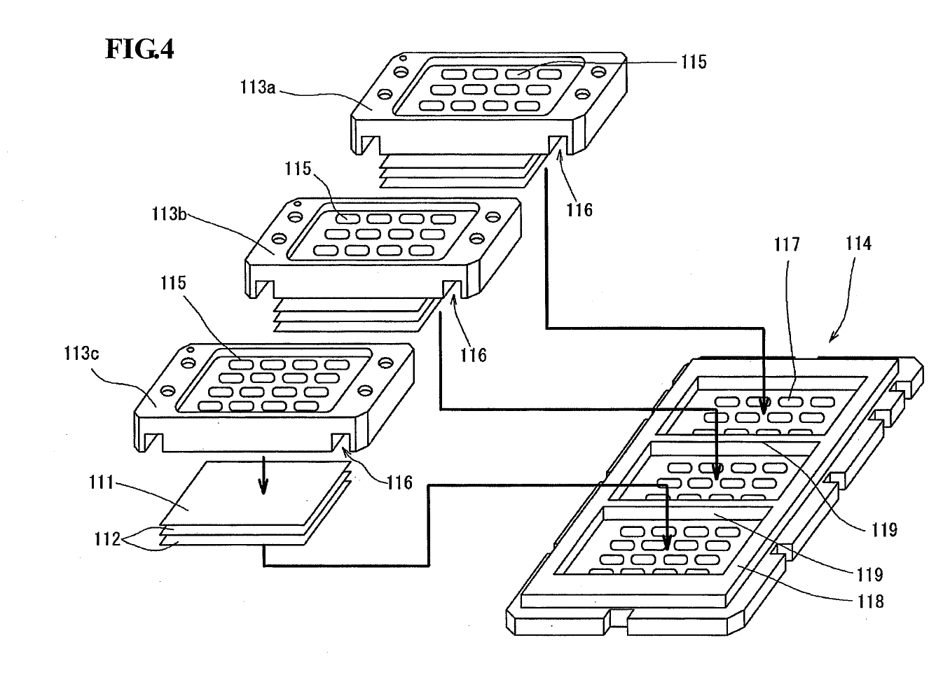

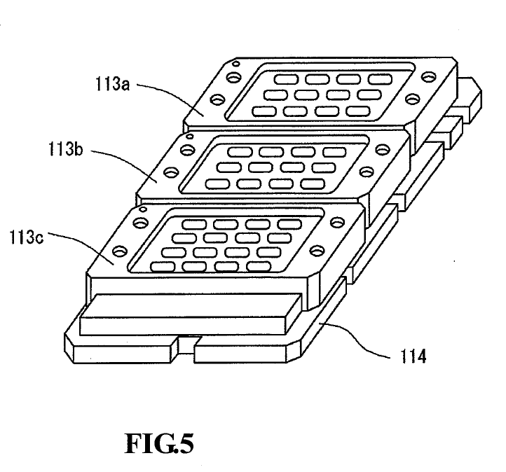

As shown in FIGS. 4 and 5, the solid phase tip for protein 101 is configured by a porous film 111 and a filter paper 112, and upper plate 113 and lower plate 114 for sandwiching the porous film 111 and the filter paper 112. The solid phase tip for protein 101 has a function of contacting the antibody solution containing antibody of cyclin-dependent kinase and the biological sample (specimen).

As shown in FIGS. 4 and 5, the upper plate 113 is configured by three plates independent from each other, that is, a first upper plate 113a, a second upper plate 113b, and a third upper plate 113c. Each upper plate has a rectangular plate shape, wherein the first upper plate 113a and the second upper plate 113b are both perforated with twelve oval through holes 115 arrayed in a matrix form of four by three, and the third upper plate 113c is perforated with sixteen oval through holes 115 arrayed in a matrix form of four by four. Each upper plate includes a region, which is independent from each other for sample processing, formed with a plurality of through holes. A groove 116 is formed along a short side at the bottom surface of each upper plate.

A total of forty oval through holes 117 arrayed in a matrix form is formed in the lower plate 114 having a rectangular plate shape at positions corresponding to each through hole 115 of the upper plates 113a, 113b, 113c. The through holes 117 have the same shape and cross sectional area as the through holes 115. The lower plate 114 has a region formed with a plurality of through holes corresponding to each region of the upper plates 113a, 113b, 113c.

A rib-shaped convex part 118 which goes around the periphery of the forty through holes 117 once, and a partition wall 119 for partitioning the through holes 117 to three regions in correspondence to each region of the upper plate 113a, 113b, 113c are formed on the upper surface of the lower plate 114. Three rectangular porous film installing regions are partitioned on the inner side by the convex part 118 and the partition wall 119. The upper plate 113 and the lower plate 114 are made of vinyl chloride resin and the like.

As shown in FIGS. 2 to 5, a stacked body including the porous film 111 and the filter paper (filter) 112 is mounted on the porous film installing region of the lower plate 114, and the grooves 116 of each upper plate 113a, 113b, 113c are sequentially fitted to the convex part 118 of the lower plate 114, so that the upper plates 113a, 113b, 113c area attach the lower plate 114 thereby forming the solid phase tip for protein 101. Each through hole 115 and each through hole 117 then become coaxial to each other.

The solid phase tip for protein described above has the upper plate divided into three, so that three regions can be aspirated independently. The number of upper plates may be two, or four or more, and is not particularly limited in the present invention. The number of upper plates is appropriately selected in view of the number of measurement items and the number of specimens.

[Activity Measuring Sample Preparation Unit]

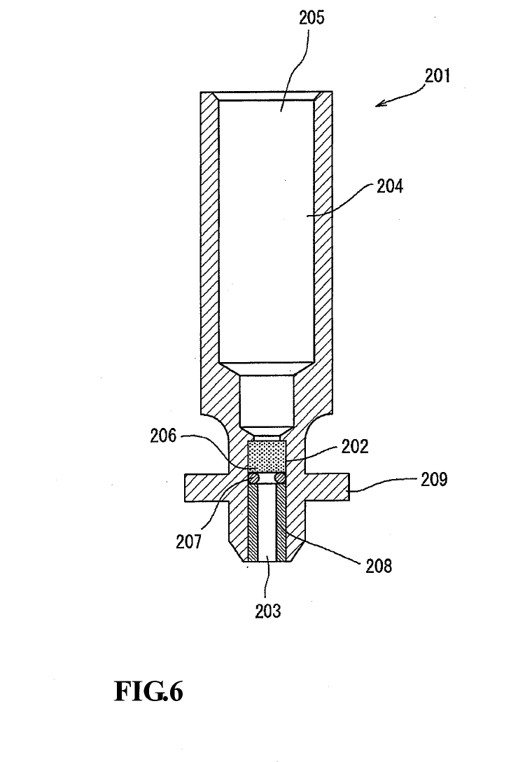

As shown in FIGS. 6 to 10, the activity measuring sample preparation unit 2 includes a plurality of sample preparation units 211 each including a column 201 and a fluid manifold 213, and is used to measure the activity value of the CDK.

The column 201 shown in FIG. 6 is made of a cylindrical body made of vinyl chloride resin, and includes therein a carrier holding member 202 for holding a carrier 206 used to isolate the target substance in the liquid sample, and a liquid storage member 204 for receiving and storing the liquid sample to introduce the liquid sample to the carrier holding member 202. The column 201 has an opening 205 through which the liquid sample is externally injected or from which the liquid sample is collected at the upper part of the liquid storage member 204, and includes a connection flow channel 203 for introducing the liquid sample to the fluid manifold 213 and receiving the liquid sample from the fluid manifold 213 at the lower part of the carrier holding member 202. The column 201 configures a means for contacting the substrate solution containing a predetermined substrate with the biological sample (specimen).

The carrier 206 is made of monolithic silica gel of circular cylinder shape, wherein the monolithic silica gel has a configuration in which the three-dimensional network frame work and the void thereof are integrated. The predetermined CDK antibody is immobilized to the monolithic silica gel. The carrier 206 is inserted to the carrier holding member 202 from the lower opening of the column 201, and is elastically pushed and supported by a fixing pipe 208 by way of an O-ring 207. The fixing pipe 208 is press-fit from the lower opening of the column 201, wherein the hole of the fixing pipe 208 and the O-ring 207 form the connection flow channel 203.

A mounting flange 209 for mounting and fixing the column 201 to the sample preparation unit 211 is formed at the lower end of the column 201. The flange 209 is an oval flange formed by cutting out both sides of a disc shaped flange having a diameter D in parallel so as to have a width W (W<D).

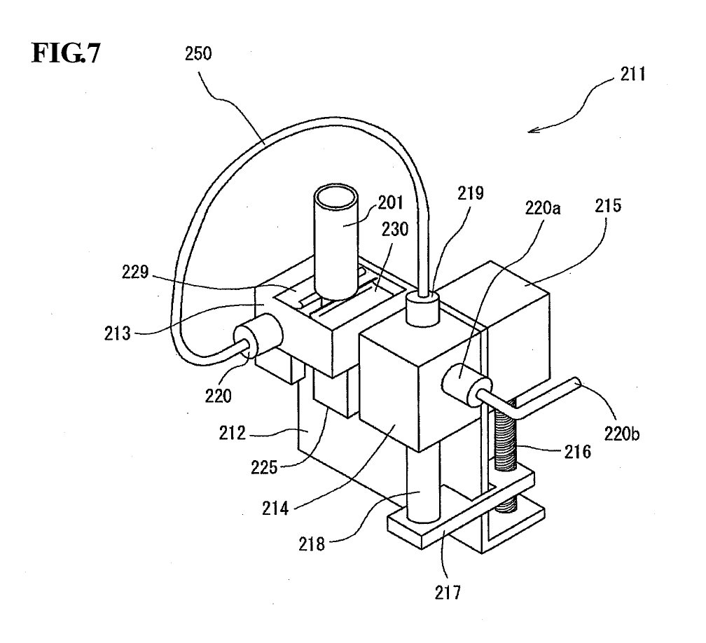

FIG. 7 is a perspective view of the sample preparation unit 211, wherein the sample preparation unit 211 includes an L-shaped supporting plate 212, and the fluid manifold 213, a syringe pump 214, and a stepping motor with reducer 215 are fixed on the supporting plate 212, as shown in the figure.

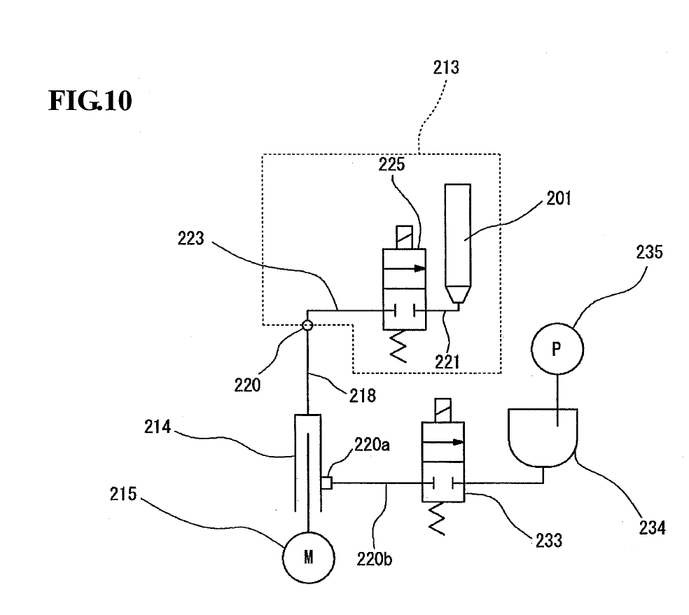

A screw shaft 216 is connected to the output shaft of the stepping motor 215. A drive arm 217 to be screw fit to the screw shaft 216 is connected to the distal end of a piston 218 of the syringe pump 214. The piston 218 moves up and down when the screw shaft 216 is rotated by the stepping motor 215. The syringe pump 214 and the fluid manifold 213 are connected to a liquid feeding tube 250 by way of connectors 219, 220. The syringe pump 214 is connected to a chamber 234 (see FIG. 10) accommodating fluid (washing liquid) for filling the flow channel by a liquid feeding tube 220b by way of a connector 220a.

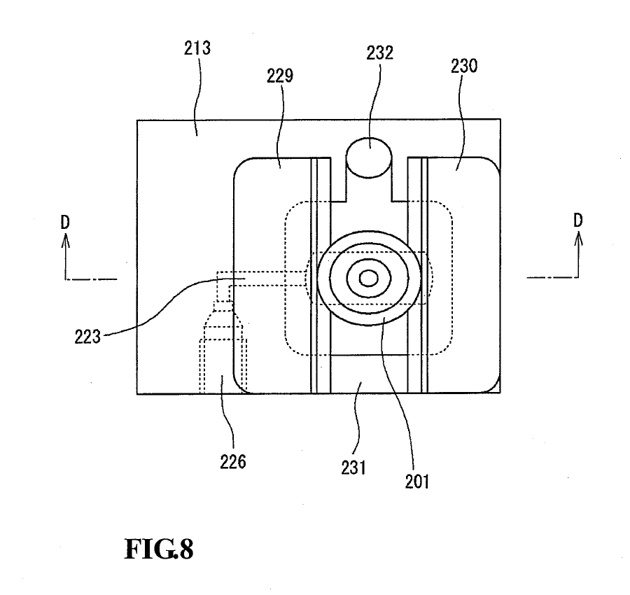

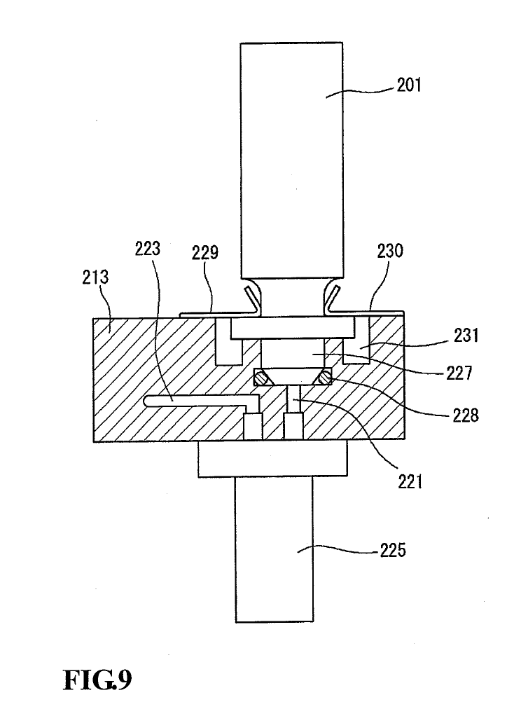

As shown in FIGS. 8 and 9, the fluid manifold 213 includes a column connecting part 221 to which the lower opening of the column 201 is connected.

The fluid manifold 213 includes a flow channel 223 therein, and has an electromagnetic valve 225 on the lower surface for opening/closing between the flow channel 223 and the column connecting part 221. The fluid manifold 213 has a connector connection screw hole 226 for connecting a connector 220 on the side surface, wherein the screw hole 226 is connected to the flow channel 223.

FIG. 10 is a fluid circuit diagram of the sample preparation unit 211, wherein a state in which the syringe pump 214 is connected to the fluid manifold 213 by way of the connector 220 is shown. A chamber 234 is connected to the syringe pump 214 by way of the electromagnetic valve 233, and positive pressure is applied to the chamber 234 from a positive pressure source 235.

A method of mounting the column 201 to the fluid manifold 213 will now be described.

As shown in FIGS. 8 to 10, a column mounting concave part 227 for receiving the lower end of the column 201 is formed on the upper surface of the fluid manifold 213, the center of the bottom part of the concave part 227 passing through the column connecting part 221, and an O-ring 228 being attached to the circumference of the bottom part. Two pressing plates 229, 230 having a cross section of L-shape are fixed in parallel on the upper surface of the fluid manifold 213 at an interval wider than the width W and narrower than D with the column mounting concave part 227 as the center.

In order to prevent specimen or reagent that has passed the carrier 206 inside the column 201 fixed to the fluid manifold 213 from contacting fluid (washing liquid) that fills the flow channel 223 inside the fluid manifold 213 and being diluted, the electromagnetic valve 225 is opened (electromagnetic valve 233 is closed) before the column 201 is fixed to the column mounting concave part 227 and the syringe pump 214 is aspiration operated by about 16 μL. The liquid level of the column connecting part 221 thereby lowers and an air gap is forms.

Subsequently, the column 201 is mounted to column mounting concave part 227 so that the flange 209 passes between the pressing plates 229, 230, and then rotated clockwise or counterclockwise by 90 degrees. The portion of the diameter D of the flange 209 engages the pressing plates 229, 230, and the flange 209 is fixed by the pressing plates 229, 230 due to the elasticity of the O-ring 228. When removing the column 201, the column 201 is rotated either to the left or the right by 90 degrees while being pressed.

When the column 201 is mounted to fluid manifold 213 of the sample preparation unit 211, the concave part 227 of the fluid manifold 213 is filled with manually or automatically dispensed fluid to prevent mixture of air bubbles, but when the distal end of the column 201 is inserted to the concave part 227, the fluid flows out due to increase in volume. An overflow storage concave part 231 is arranged at the periphery of the column mounting concave part 227 to prevent the fluid from flowing out to the periphery, and an overflow liquid discharging concave part 232 for aspirating and discharging the overflow liquid by pipette is arranged at one part of the overflow liquid storage concave part 231.

Various specimens and reagents are injected or aspirated to or from a predetermined location by the dispensing mechanical member 3 equipped with the pipette.

The operation of the upper opening 205 of the column 201 of the case the sample or the reagent is injected will now be described. The electromagnetic valve 225 is opened (electromagnetic valve 233 is closed), and the syringe pump performs the aspirating operation when the specimen or the reagent is injected to the opening 205. The air gap and the specimen or the reagent pass through the electromagnetic valve 225, and are then aspirated to the syringe pump side. The syringe pump then performs ejecting operation. The specimen or the reagent passes through the electromagnetic valve 225, and is sent to the column 201.

[Dispensing Mechanical Member]

As shown in FIG. 1, the dispensing mechanical member 3 includes a frame 352 for moving the pipette in the X direction, a frame 353 for moving the pipette in the Y direction, and a plate 354 for moving the pipette in the Z direction.

The frame 352 includes a screw shaft 355 for moving the plate 354 in the direction of the arrow X, a guide bar 356 for supporting and slidably moving the plate 354, and a stepping motor 357 for rotating the screw shaft 355.

The frame 353 includes a screw shaft 358 for moving the plate 352 in the direction of the arrow Y, a guide bar 359 for supporting and slidably moving the frame 352, and a stepping motor 361 for rotating the screw shaft 358.

The plate 354 includes a screw shaft 367 for moving an arm 368 supporting the pipette 362 in the direction of the arrow Z, a guide bar for supporting and slidably moving the arm 368, and a stepping motor 370 for rotating the screw shaft 367.

In the present embodiment, since the dispensing mechanical member 3 is equipped with a pair of pipettes 362, reagent etc. is simultaneously injected to two specimen containers and content is simultaneously aspirated from two sample containers, whereby the measuring process can be efficiently performed.

[Fluid Unit]

As shown in FIG. 1, a fluid unit 9, connected to the pipette washing bath 8 for washing the pipette 362 and each sample preparation unit 211, for operating the fluid is arranged at the rear part of the apparatus body 20. As shown in FIG. 10, the fluid unit 9 includes an electromagnetic valve 225 of each sample preparation unit 211, an electromagnetic valve 233 for controlling the fluid when filing the liquid from the washing liquid chamber to the syringe 214, an electromagnetic valve for controlling fluid when aspirating and ejecting the liquid with the pipette 362, an electromagnetic valve for controlling the fluid when aspirating the liquid wasted from the pipette 362 in the waste bath 7, and an electromagnetic valve for controlling the fluid when washing the pipette 362 in the pipette washing bath 8.

[Detecting Unit]

The detecting unit 4 is provided to measure the fluorescent substance quantity reflecting the protein quantity and the fluorescent substance quantity reflecting the amount of phosphate group captured at the porous film 111 of the solid phase tip for protein 101, wherein excitation light is irradiated on the solid phase tip for protein 101, the generated fluorescence is detected, and the electric signal having a magnitude corresponding to the intensity of the detected fluorescence is output to the main body controller 10. A generally used detecting unit configured by light source unit, illumination system, and light receiving system is appropriately adopted for the detecting unit 4.

[Data Processing Unit]

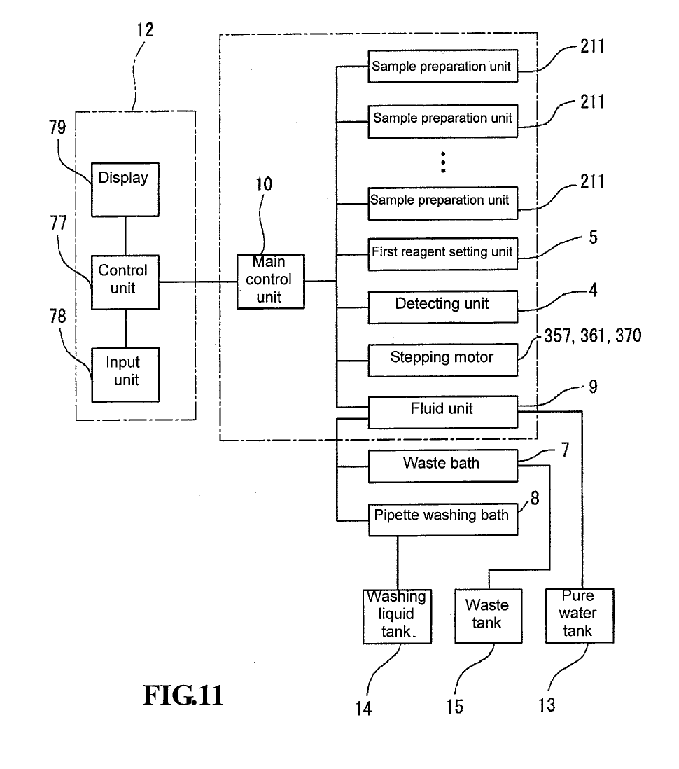

FIG. 11 is a block diagram showing a configuration of the diagnosis support apparatus A of the present embodiment. As shown in FIG. 11, the personal computer 12 which is a data processing unit includes a controller 77, an input unit 78, and a display 79.

The controller 77 has a function of transmitting an operation start signal of the apparatus to the main body controller 10 described below. When a command of operation start is transmitted from the controller 77, the main body controller 10 outputs a drive signal for driving the stepping motor 215 of each sample preparation unit 211, a drive signal for adjusting the temperature of the first reagent setting unit 5, a drive signal for driving the stepping motors 357, 361, 370, and a drive signal for driving the electromagnetic valve in the fluid unit 9. The controller 77 also has a function for analyzing the detection result obtained in the detecting unit 4. The detection result obtained in the detecting unit 4 is transmitted to the main body controller 10. The main body controller 10 transmits the detection result obtained in the detecting unit 4 to the controller 77. The controller 77 analyzes the detection result transmitted from the main body controller 10.

The display 79 is arranged to display analysis result etc. obtained in the controller.

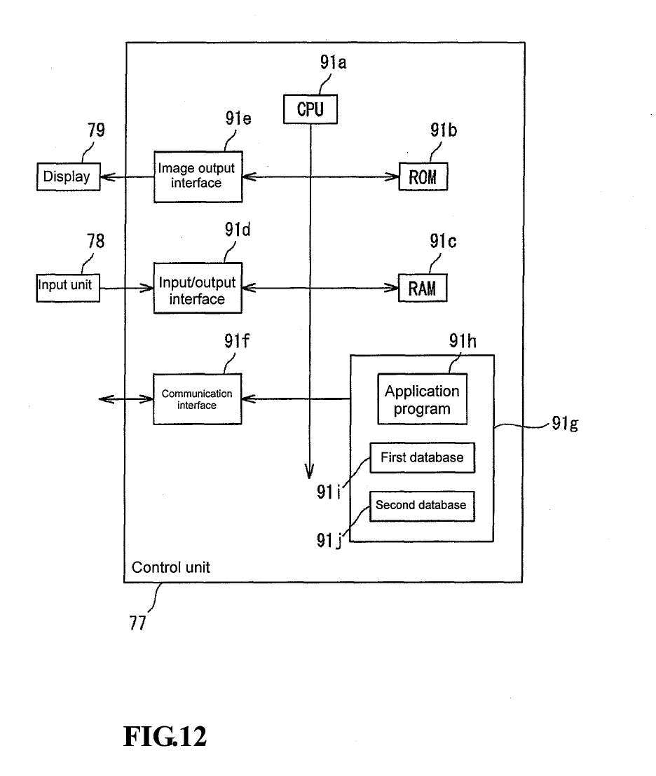

The configuration of the personal computer 12 will now be described in detail. As shown in FIG. 12, the controller 77 is mainly configured by a CPU 91a, a ROM 91b, a RAM 91c, an input/output interface 91d, an image output interface 91e, a communication interface 91f, and a hard disc 91g. The CPU 91a, the ROM 91b, the RAM 91c, the input/output interface 91d, the image output interface 91e, the communication interface 91f, and the hard disc 91g are connected with an electric signal line (bus) so as to communicate electrical signals.

The CPU 91a executes computer programs stored in the ROM 91b and the computer programs loaded in the RAM 91c. The personal computer 12 serves as the data processing unit when the CPU 91a executes the application program 91h, as hereinafter described.

The ROM 91b is configured by mask ROM, PROM, EPROM, EEPROM, and the like, and is recorded with computer programs executed by the CPU 91a, data used for the same, and the like.

The RAM 91c is configured by SRAM, DRAM, and the like. The RAM 91c is used to read out the computer programs recorded on the ROM 91b and the hard disc 91g. The RAM 91c is used as a work region of the CPU 91a when executing the computer programs.

Various computer programs executed by the CPU 91a such as operating system and application program, and data used in executing the computer program are installed in the hard disc 91g. The application program 91h for acquiring the expression level and the activity value of the cyclin-dependent kinase, calculating the ratio of the activity value and the expression level, calculating a ratio (specific activity ratio) of the ratio of the first cyclin-dependent kinase and the ratio of the second cyclin-dependent kinase, and comparing the calculated ratio and the specific activity ratio with the reference value to acquire diagnosis support information of cancer is also installed in the hard disc 91g, according to the present embodiment. In order to acquire the expression level and the activity value, standard curve of conversion data for converting fluorescence intensity to expression level or activity value is stored in the hard disc 91g. The standard curve may be obtained for every measurement of the expression level or the activity value. The hard disc 91g includes a first database 91i for storing the reference value for acquiring the diagnosis support information of the cancer by being compared with the measurement value. The hard disc 91g also includes a second database 91j for storing sample data in which the measurement value such as the activity value and the expression level of the cancer patient and the clinical information of the patient are corresponded to each other.

Operating system providing graphical user interface environment such as Windows (registered trademark) manufactured and sold by US Microsoft Co. is installed in the hard disc 91g. In the following description, the application program 91h according to the present embodiment is assumed to operate on the operating system.

The input/output interface 91d is configured by serial interface such as USB, IEEE1394, RS-232C; parallel interface such as SCSI, IDE, IEEE1284; analog interface such as D/A converter, A/D converter, and the like. The input unit 78 is connected to the input/output interface 91d, so that the user can input data to the personal computer 12 using the input unit 78.

The communication interface 91f is, for example, Ethernet (registered trademark) interface. The personal computer 12 transmits and receives data with the main body controller 10 using a predetermined communication protocol by means of the communication interface 91f.

The image output interface 91e is connected to the display 79 configured by LCD, CRT, or the like, and is configured to output an image signal corresponding to the image data provided from the CPU 91a to the display 79. The display 79 displays the image (screen) according to the input image signal.

[Main Body Controller]

The main body controller 10, connected to each sample preparation unit 211, the detecting unit 4, the stepping motors 357, 361, 370, the fluid unit 9 and the like, for controlling the same is arranged at a back part of the apparatus body 20.

As shown in FIG. 13, the main body controller 10 includes a CPU 301a, a ROM 301b, a RAM 301c, a communication interface 301d, and a circuit part 301e.

The CPU 301a executes computer programs stored in the ROM 301b and the computer programs read out the RAM 301c.

The ROM 301b stores a computer program for allowing the CPU 301a to execute, data used in the execution of the computer program, and the like.

The RAM 301c is used in reading out the computer program stored in the ROM 301b. The RAM 301c is used as a work region of the CPU 301a when executing the computer programs.

The communication interface 301d is, for example, Ethernet (registered trademark) interface. The main body controller 10 transmits and receives data with the personal computer 12 using a predetermined communication protocol by means of the communication interface 301d.

The circuit part 301e includes a plurality of drive circuits and a signal processing circuit (not shown). The drive circuit is arranged in correspondence to the sample preparation unit 211, the first reagent setting unit 5, the detecting unit 4, the stepping motors 357, 361, 370, and the fluid unit 9. Each drive circuit generates a control signal (drive signal) for controlling the corresponding unit (sample preparation unit 211 if drive circuit corresponding to the sample preparation unit 211) according to the instruction data provided from the CPU 301a, and transmits the control signal to the unit. The output signal of the sensor arranged in the unit is provided to the drive circuit, wherein the drive circuit converts the output signal to a digital signal and provides the same to the CPU 301a. The CPU 301a generates the instruction data based on the provided output signal of the sensor.

The signal processing circuit is connected to the detecting unit 4. A detection signal indicating fluorescence intensity is output from the detecting unit 4, and such detection signal is provided to the signal processing circuit. The signal processing circuit executes signal processes such as noise removal process, amplification process, and A/D conversion process on the detection signal. The data on the detection result obtained as a result of signal process is provided to the CPU 301a.

[3] Diagnosis Support of Cancer

An example of the case of determining the malignancy (possibility of recurrence risk) of the cancerous cell of a human using the diagnosis support apparatus A according to the present embodiment will now be described.

(1) Pre-Process by Solubilizing Device B

Prior to the process by the diagnosis support apparatus A, liquid specimen is collected from the tissue containing the malignant tumor extirpated from a cancer patient using the solubilizing device B. In the procedure thereof, the tissue is first placed in an eppen tube using a pin set. The eppen tube is then set in the sample setting member 33 of the solubilizing device B shown in FIG. 1, and the start button of the operating member 31 is pushed, whereby the pestle 34 lowers to a predetermined position and pushes the tissue in the eppen tube against the bottom of the eppen tube.

Solubilizing liquid such as buffer solution containing surfactant and proteolysis enzyme inhibitor is automatically or manually injected into the eppen tube in such state. Thereafter, the pestle 34 is rotated to grind the tissue. The drive of the pestle 34 is stopped after a predetermined time has elapsed, the pestle 34 is moved upward, and thereafter, the eppen tube is taken out from the sample setting member 33. The solubilized content in the eppen tube is then set in the centrifugal machine, and the obtained supernatant solution is manually collected as a specimen.

(2) Setting of Specimen Etc. to the Diagnosis Support Apparatus A

The supernatant solution is placed in two specimen containers and diluted at dilution ratio different from each other, and thereafter, the specimen containers are set at predetermined positions in the first reagent setting unit 5. Of the two specimens, one is the specimen for expression level measurement, and the other is the specimen for activity value measurement.

The solid phase tip for protein 101 is set in the chip setting unit 1, and eight columns 201 are respectively set in the sample preparation unit 211 of the activity measurement unit 2.

(3) Overall Flow of Process by Diagnosis Support Apparatus A (First Embodiment)

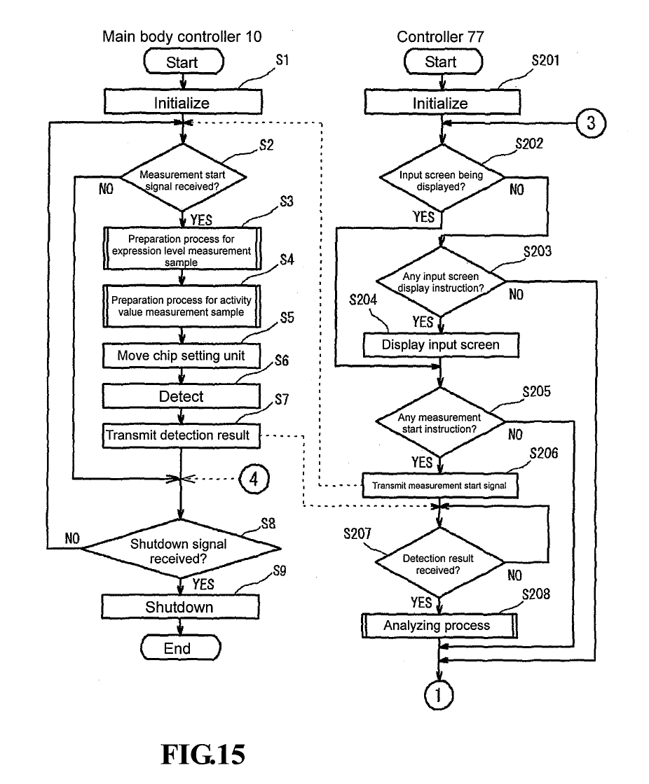

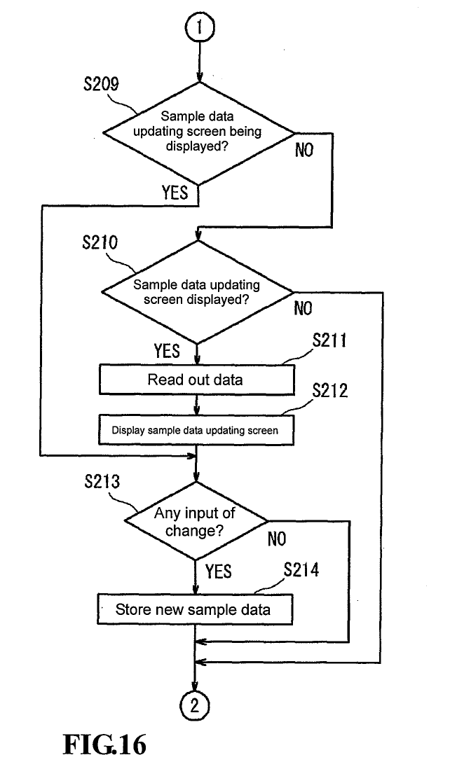

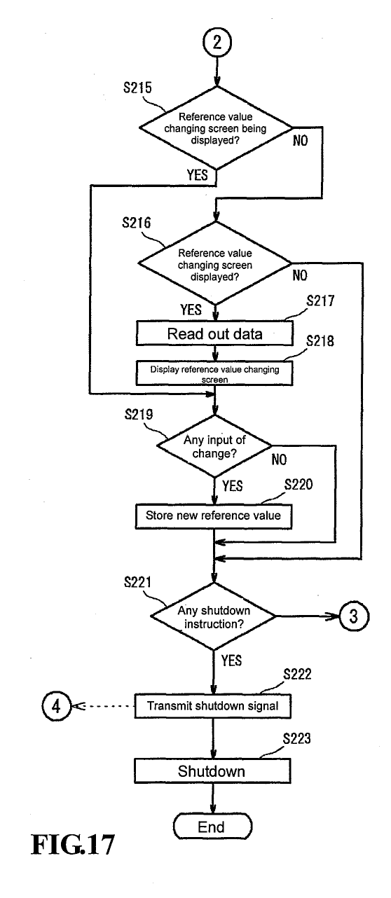

The overall flow of the process according to the first embodiment by the diagnosis support apparatus A is shown in FIGS. 15, 16, and 17. In the judgment in the following flowchart, down refers to Yes and right (left) refers to No unless specifically written as “Yes” and “No”. The processes described below are all processes controlled by the controller 77 and the main body controller 10.

When the power of the apparatus body 20 is turned ON, initialization of the main body controller 10 is performed (step S1). In this initialization operation, initialization of the program, return to an origin position for the driving member of the apparatus body 20, and the like are performed.

When the power of the personal computer 12 is turned ON, initialization of the controller 77 is performed (step S201). In this initialization operation, initialization of the program, or the like is performed. After the initialization is completed, a menu screen (not shown) including an input screen display button for instructing the display of an input screen is displayed on the display 79. The user operates the input unit 78 to select the input screen button for instructing the display of the input screen of the menu screen.

In step S202, the controller 77 determines whether or not the input screen is being displayed. The controller 77 advances the process to step S205 if determined that the input screen is being displayed (Yes), and advances the process to step S203 if determined that the input screen is not being displayed (No).

In step S203, the controller 77 determines whether or not a display instruction of the input screen has been made (whether or not input screen button for instructing the display of the input screen of the menu screen is selected). The controller 77 advances the process to step S204 if determined that the display instruction of the input screen has been made (Yes), and advances the process to step S209 if determined that the display instruction of the input screen has not been made (No).

In step S204, the controller 77 displays the input screen on the display 79. The user operates the input unit 78 to input information related to the measurement such as specimen number. When the user selects the start button displayed on the input screen by operating the input unit 78, the instruction to start the measurement is made.

In step S205, the controller 77 determines whether or not the instruction to start the measurement is made. The controller 77 advances the process to step S206 if determined that the instruction to start the measurement is made (Yes), and advances the process to step S209 if determined that the instruction to start the measurement is not made (No). In step S206, a measurement start signal is transmitted from the controller 77 to the main body controller 10.

In step S2, the main body controller 10 determines whether or not the measurement start signal is received. The main body controller 10 advances the process to step S3 if determined that the measurement start signal is received (Yes), and advances the process to step S8 if determined that the measurement start signal is not received (No).

In step S3, the sample for expression level measurement is prepared. The specimen is aspirated from the specimen container set in the first reagent setting unit 5. A predetermined process is performed on the aspirated specimen, and the sample for expression level measurement is prepared.

In step S4, the sample for activity value measurement is prepared. The specimen is aspirated from the specimen container set in the first reagent setting unit 5. A predetermined process is performed on the aspirated specimen, and the sample for activity value measurement is prepared.

In step S5, the chip setting unit 1 set with the solid phase tip for protein 101 including the sample for expression level measurement and the sample for activity value measurement is moved from the position shown in FIG. 1 into the detecting unit 4.

In step S6, excitation light is irradiated on each well of the solid phase tip for protein 101, and fluorescence radiated from each sample is detected.

In step S7, the detected detection result is transmitted from the main body controller 10 to the controller 77.

In step S207, the controller 77 determines whether or not the detection result is received. The controller 77 advances the process to step S208 if determined that the detection result is received (Yes). The controller 77 again executes the process of step S207 if determined that the detection result is not received (No).

In step S208, the controller 77 executes an analyzing process from the acquired detection result, and outputs the analysis result.

In step S209, the controller 77 determines whether or not a sample data updating screen is displayed. The controller 77 advances the process to step S213 if determined that the sample data updating screen is displayed (Yes), and advances the process to step S210 if determined that the sample data updating screen is not displayed (No).

In step S210, the controller 77 determines whether or not the display instruction of the sample data updating screen has been made. The controller 77 advances the process to step S211 if determined that the display instruction of the sample data updating screen has been made (Yes), and advances the process to step S215 if determined that the display instruction of the sample data updating screen has not been made (No).

In step S211, the RAM 91c of the controller 77 reads out the sample data stored in the second database 91j of the hard disc 91g.

In step S212, the sample data updating screen including the read sample data is displayed on the display 79. The user operates the input unit 78 to select the item to be changed, and information after change is input for the selected item.

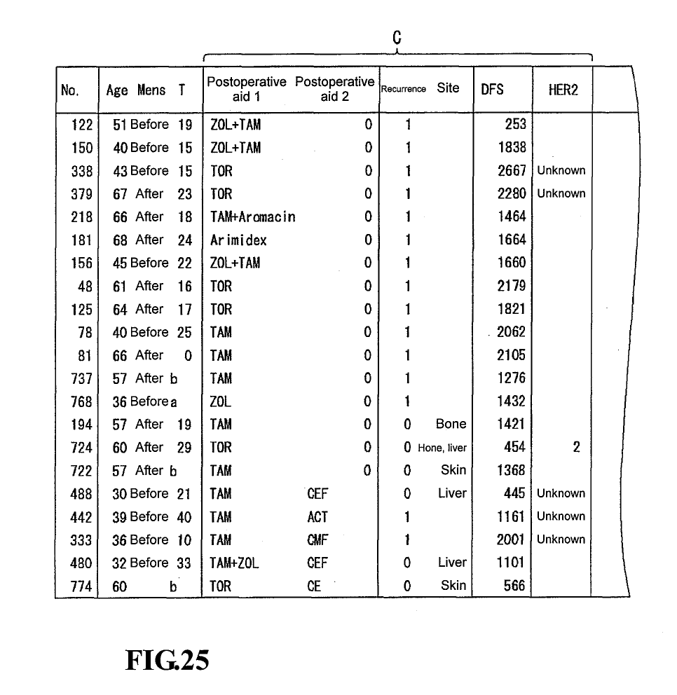

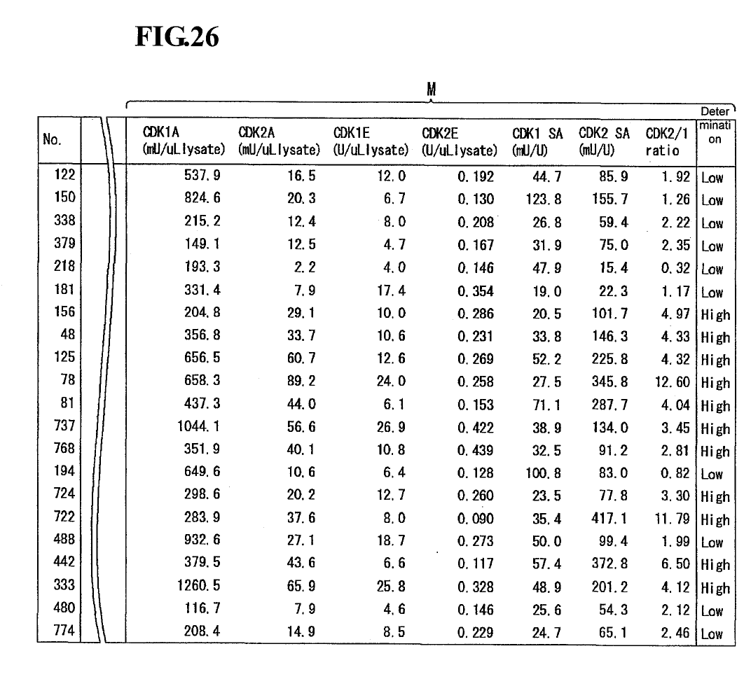

The sample data updating screen is configured so that measurement value, clinical information, and the like of the patient stored in the second database 91j as sample data are displayed in a list form as shown in FIGS. 25 and 26. In this case, all the measurement values and clinical information stored with respect to the patient can be displayed, but information makes it difficult to see and operate the screen if the number of items becomes too large, and thus the display item selection screen may be displayed so that the user (operator) can select the items displayed after the display instruction of the sample updating screen is made in step S210. In the present embodiment, examples of displayed items include presence of lymph node metastasis, presence of recurrence, number of days from extirpation until recurrence, expression level, activity value, and specific activity of CDK1 and CDK2, and specific activity ratio.

In step S213, the controller 77 determines whether or not input of new sample data is made. The controller 77 advances the process to step S214 if determined that input of the new sample data is made (Yes), and advances the process to step S215 if determined that input of the new sample data is not made (No).

In step S214, the input new sample data is stored in the second database 91j of the hard disc 91g.

In step S215, the controller 77 determines whether or not a reference value updating screen is being displayed. The controller 77 advances the process to step S219 if determined that the reference value updating screen is being displayed, and advances the process to step S216 if determined that the reference value updating screen is not being displayed (No).

In step S216, the controller 77 determines whether or not a display instruction of the reference value updating screen has been made. The controller 77 advances the process to step S217 if determined that the display instruction of the reference value updating screen has been made (Yes), and advances the process to step S221 if determined that the display instruction of the reference value updating screen has not been made (No).

In step S217, the RAM 91c of the controller 77 reads out the sample data stored in the second database 91j and the reference value stored in the first database 91i of the hard disc 91g.

In step S218, the reference value updating screen including a graph created based on the read sample data and reference value is displayed on the display 79.

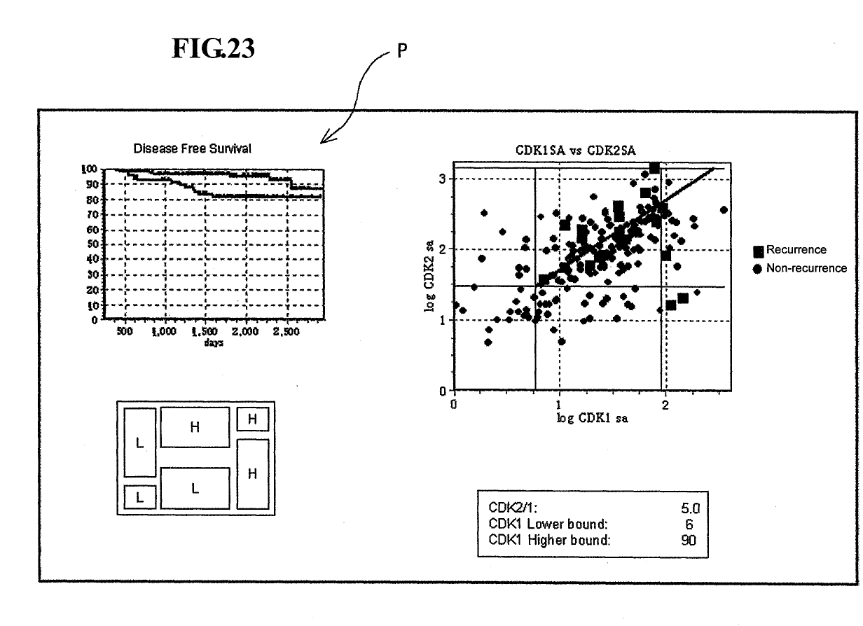

FIG. 23 shows an example of a reference value changing screen (before change). In this example, the specific activity of the CDK1, and the ratio of the specific activity of the CDK1 and the specific activity of the CDK2 are selected as a reference for determining the recurrence risk. Regarding the specific activity of the CDK1, a second reference value (=6) which is a low level reference value and a third reference value (=90) which is a high level reference value are set, and a reference value (first reference value) of the ratio of the specific activities is set to “5.0”. Six regions partitioned by such three reference values are set to “high (H)” or “low (L)” region, as shown on the left lower of FIG. 23.

The graph P on the left upper of the FIG. 23 is a survival curve having the horizontal axis as the number of days after the operation and the vertical axis as a non-recurrence rate (unit in %), showing the recurrence rate of “high” region and “low” region. The curve on the upper side indicates the recurrence rate of recurrence risk “low” region, and the curve on the lower side indicates the recurrence rate of recurrence risk “high” region. This graph is created in the following manner.

As described above, after the setting of the reference value and the setting of the region (setting of “high” region or “low” region), the controller 77 performs the calculation of the non-recurrence rate for every region. The sample data contains information (history information) related to the presence of recurrence, and the number of days (elapsed number of days after operation for patients without recurrence) from the extirpative operation until recurrence. The non-recurrence rate is calculated with all the sample data contained in each region as population and the number of days after the operation as a variable, and the result thereof is formed into a graph. For simplicity, assume the number of samples within the “low” region as 100, sample 1, sample 2, and sample 3 recurs in 600 days, 900 days, and 1200 days after the operation, and the remaining samples does not recur for five years (about 1826 days) after the operation. In this case, the non-recurrence rate up to 600 days after the operation is 100(%), and 99(%) at 600 days after the operation, and remains 99(%) up to 900 days after the operation. At 900 days after the operation, the non-recurrence rate becomes 98(%), and remains 98% up to 1200 days after the operation. At 1200 days after the operation, the non-recurrence rate becomes 97(%), and remains 97% up to 1826 days after the operation. That is, the recurrence risk for five years after the operation is 3%. If the patient dies for reasons other than cancer without cancer recurring after the operation, the patient is deleted from the population of sample data at the time of death. Regarding the number of days until recurrence after the extirpative operation, the number of days can be known through health check and examination for patients with recurrence when the patient comes to the hospital. For other patients, the hospital contacts the patient for follow up on a regular basis, and determines as “no recurrence” for patients answering “normal”. The patients who have not answered to the follow up or patients who are no longer the target of follow up are deleted from the population of the sample data. Therefore, as the number of days after the operation elapses, the number of population gradually decreases, and the proportion in lowering of the non-recurrence rate due to recurrence in one patient becomes larger.

The reference value is changed by the user by operating the input unit 78, placing the pointer on a linear line representing the reference value in the graph and dragging the pointer, or arranging a scroll bar or a button (not shown) in the screen, and operating the same by the user.

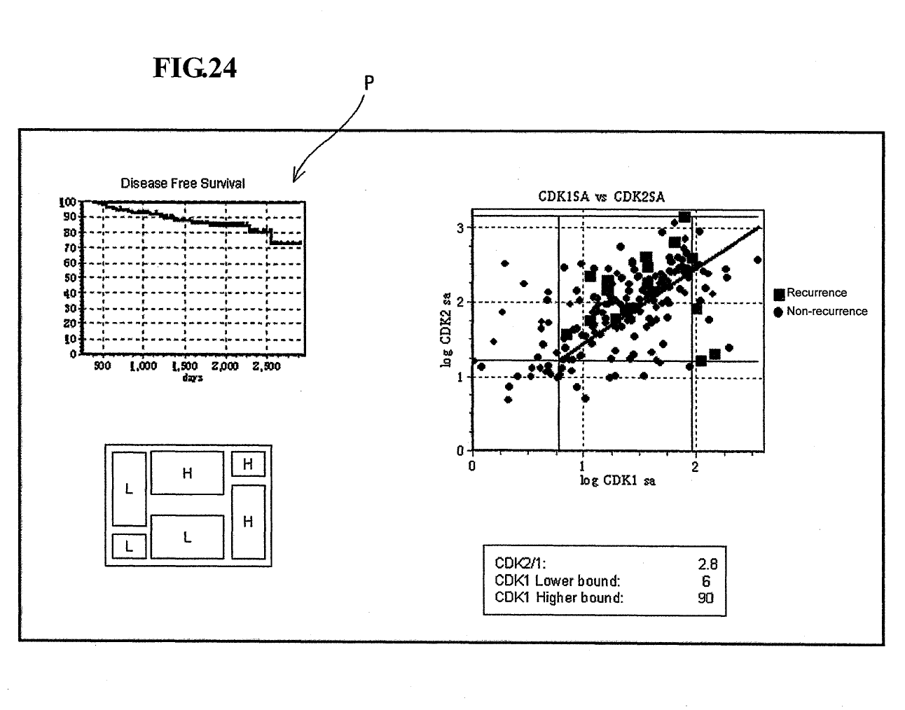

FIG. 24 shows a screen after the reference value is changed, wherein the first reference value (ratio of specific activity) is changed from 5.0 to 2.8. The “high” region and “low” region of the recurrence risk also change by changing the reference value, but in the present embodiment, when the reference value is changed, the controller 77 creates the survival curve based on the sample data contained in the region partitioned by the changed reference value and displays it on the display 79. Therefore, the user can appropriately change the reference value so that the recurrence rate after elapse of five years takes a value within a predetermined range while looking at the survival curve, and as a result, can set a reference value having a high determination accuracy. Only the curve indicating the recurrence rate of recurrence risk “high” region seems to be present in the graph P showing the survival curve in FIG. 24. This is because, an event in which the patient of recurrence risk “low” region recurs is not contained as a result of changing the reference value, the non-recurrence rate of recurrence risk “low” region remains 100%, and the curve overlaps the line indicating 100% in the horizontal axis, and thus in reality, two curves are displayed. In the example shown in FIG. 24, only the first reference value is changed, but the second reference and/or third reference value may be changed.

In step S219, the controller 77 determines whether or not an input to change the reference value is made. The controller 77 advances the process to step S220 if determined that an input of the new reference value is made (Yes), and advances the process to step S221 if determined that an input of the new reference value is not made (No).

In step S220, the input new reference value is stored in the first database 91i of the hard disc 91g.

In step S221, the controller 77 determines whether or not an instruction to shutdown is accepted. The controller 77 advances the process to step S222 if determined that the instruction to shutdown is accepted (Yes), and returns the process to step S202 if determined that the instruction to shutdown is not accepted (No). In step S222, a shutdown signal is transmitted from the controller 77 to the main body controller 10. In step S223, the controller 77 performs the process of shutting down the personal computer 12, and completes the process.

In step S8, the main body controller 10 determines whether or not the shutdown signal has been received. The main body controller 10 advances the process to step S9 if determined that the shutdown signal has been received (Yes), and returns the process to step S2 if determined that the shutdown signal has not been received (No). In step S9, the main body controller 10 shuts down the apparatus body 20, and terminates the process.

(4) Preparation Process of Expression Level Measurement Sample

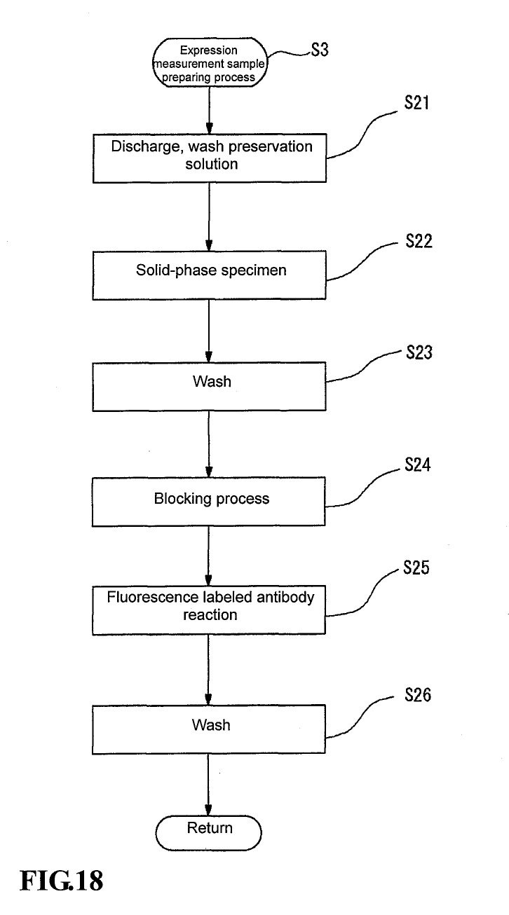

The flow of the preparation process of the expression level measurement sample in step S3 is shown in FIG. 18.

First, in step S21, the preservation solution stored in advance in each well of the solid phase tip for protein is discharged, and the inside of each well is washed. The washing is performed by injecting washing liquid to each well from the upper side through the pipette of the dispensing mechanical member 3, and aspirating the injected washing liquid through the porous film by negative pressure from the lower side of the solid phase tip for protein. The following washing step is similarly carried out.

The specimen for the expression level measurement is aspirated by the pipette from the specimen container set in the first reagent setting unit 5, the specimen is injected to a plurality of predetermined wells, and is aspirated by negative pressure from the lower side of the solid phase tip for protein. The protein is solid-phased at the porous film of the solid phase tip for protein (step S22).

Similar to step S21, the inside of the predetermined well is washed with the washing liquid. Accordingly, the components other than the protein are removed from the porous film of the solid phase tip for protein (step S23).

Subsequently, the blocking liquid is injected to the predetermined well, and after leaving it for 15 minutes or longer (e.g., for 30 minutes), the blocking liquid remaining in the well is discharged (step S24). Accordingly, the fluorescence labeled CDK1 antibody (fluorescence labeled CDK1 antibody) and the fluorescence labeled CDK2 antibody (fluorescence labeled CDK2 antibody) are prevented from being solid-phased at the site of the porous film at which the protein is not solid-phased. The commercially available fluorescence labeled CDK1 antibody and the fluorescence labeled CDK2 antibody may be used.

The fluorescence labeled CDK1 antibody and the fluorescence labeled CDK2 antibody are respectively injected to the predetermined well. In this case, each fluorescence labeled antibody is injected into two wells. After 20 to 30 minutes have elapsed, the injected fluorescence label is discharged after the reaction of the fluorescence labeled antibody and the protein (CDK1 or CDK2) solid-phased on the porous film is terminated (step S25).

Lastly, similar to step S23, the inside of the predetermined well is washed with the washing liquid (Step S26).

(5) Preparation Process of Activity Value Measurement Sample

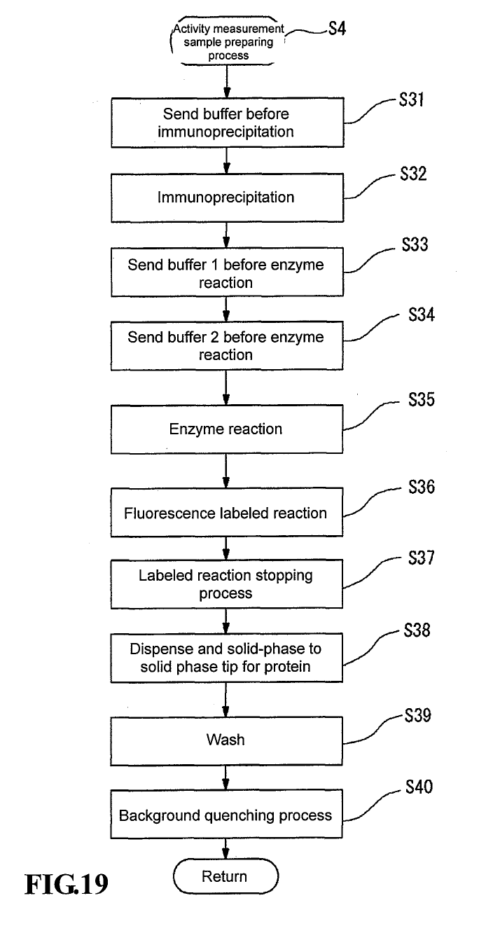

FIG. 19 shows a flow of the preparation process of the activity value measurement sample in step S4. In the preparation process of the activity value measurement sample, four sample preparation units 211 are arranged on the near side in the figure and four sample preparation units 211 are arranged on the far side in the figure as the activity measurement unit 2 shown in FIG. 1. Each sample preparation unit 211 of the activity measurement unit 2 includes a first sample preparation unit (Ac1), a second sample preparation unit (Ac2), a third sample preparation unit (Ac3), a fourth sample preparation unit (Ac4), from the left on the far side of the figure, and a fifth sample preparation unit (Ac5), a sixth sample preparation unit (Ac6), a seventh sample preparation unit (Ac7), and an eighth sample preparation unit (Ac8), from the left on the near side of the figure.

For each of the first to the eighth sample preparation units (Ac1 to Ac8), a buffer which is a washing reagent is injected to the opening 205 with the pipette of the dispensing mechanical member 3. For each of the first to the eighth sample preparation units (Ac1 to Ac8), the syringe pump 214 and the electromagnetic valve 225 operate as described above, so that the buffer of the liquid storage member 204 passes through the carrier 206 into the flow channel 223, and again passes through the carrier 206 and returns to the liquid storage member 204. The buffer returned to the liquid storage member 204 in all the columns 201 is aspirated and discharged by the pipette of the dispensing mechanical member 3 (step S31).

Immunoprecipitation (reaction between antibody and CDK) is then performed (step S32). First, the specimen 1 for the activity value measurement is aspirated with one pipette and the specimen 2 for the activity value measurement is aspirated with another pipette from one specimen container set in the first reagent setting unit 5.

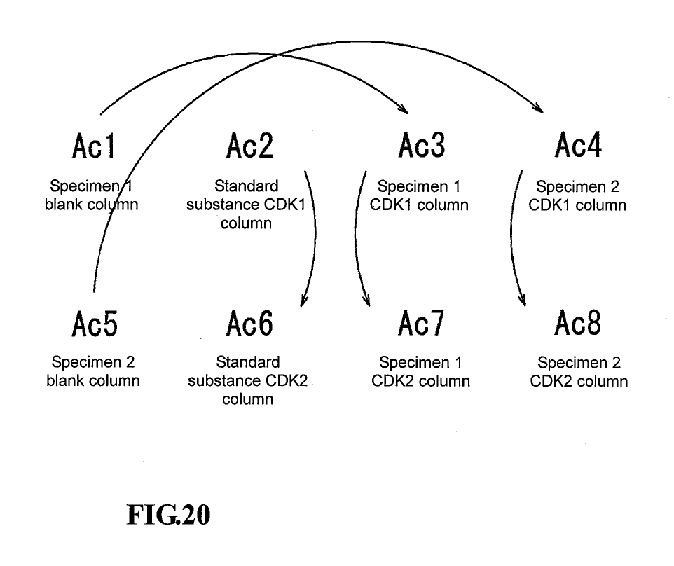

As shown in FIG. 20, the specimen 1 for the activity value measurement aspirated from the specimen container is first injected to the liquid storage member 204 of the first sample preparation unit (Ac1). The specimen 1 is sent to the carrier 206 of the first sample preparation unit (Ac1) by operating the syringe pump 214 and the electromagnetic valve 225 as described above. In this case, the specimen 1 reciprocates in the carrier 206 of the column 201 once by reciprocating the piston up and down once (aspiration→discharge).

The specimen 2 for activity value measurement aspirated from the specimen container is first injected to the liquid storage member 204 of the fifth sample preparation unit (Ac5). The specimen 2 is similarly sent to the carrier 206 of the fifth sample preparation unit (Ac5).

The antibody of the CDK1 nor the antibody of the CDK2 are immobilized on the carrier 206 of the columns 201 of the first sample preparation unit (Ac1) and the fifth sample preparation unit (Ac5). Therefore, the CDK1 and the CDK2 are not solid-phased in the first sample preparation unit (Ac1) and the fifth sample preparation unit (Ac5), the specimen 1 containing the CDK1 and the CDK2 is stored in the column 201 of the first sample preparation unit (Ac1), and the specimen 2 containing the CDK1 and the CDK2 is stored in the column 201 of the fifth sample preparation unit (Ac5).

The specimen 1 stored in the column 201 of the first sample preparation unit (Ac1) is then aspirated by the pipette, and injected to the liquid storage member 204 of the third sample preparation unit (Ac3). The specimen 1 is then sent to the carrier 206 of the third sample preparation unit (Ac3), similar to the above.

The specimen 2 stored in the column 201 of the fifth sample preparation unit (Ac5) is aspirated by the pipette, and injected to the liquid storage member 204 of the fourth sample preparation unit (Ac4). The specimen 2 is then sent to the carrier 206 of the fourth sample preparation unit (Ac4), similar to the above.

The antibody of the CDK1 is immobilized to the carriers 206 of the columns 201 of the third sample preparation unit (Ac3) and the fourth sample preparation unit (Ac4). Therefore, the CDK1 is solid-phased but the CDK2 is not solid-phased in the third sample preparation unit (Ac3) and the fourth sample preparation unit (Ac4), the specimen 1 not containing the CDK1 but containing the CDK2 is stored in the column 201 of the third sample preparation unit (Ac3), and the specimen 2 not containing the CDK1 but containing the CDK2 is stored in the column 201 of the fourth sample preparation unit (Ac4).

The specimen 1 stored in the column 201 of the third sample preparation unit (Ac3) is then aspirated by the pipette, and injected to the liquid storage member 204 of the seventh sample preparation unit (Ac7). The specimen 1 is then sent to the carrier 206 of the seventh sample preparation unit (Ac7), similar to the above.

The specimen 2 stored in the column 201 of the fourth sample preparation unit (Ac4) is aspirated by the pipette, and injected to the liquid storage member 204 of the eighth sample preparation unit (Ac8). The specimen 2 is then sent to the carrier 206 of the eighth sample preparation unit (Ac8), similar to the above.

The antibody of the CDK2 is immobilized to the carrier 206 of the columns 201 of the seventh sample preparation unit (Ac7) and the eighth sample preparation unit (Ac8). Therefore, the CDK2 is solid-phased in the seventh sample preparation unit (Ac7) and the eighth sample preparation unit (Ac8), and thus the specimen 1 not containing the CDK1 nor the CDK2 is stored in the column 201 of the seventh sample preparation unit (Ac7), and the specimen 2 not containing the CDK1 nor the CDK2 is stored in the column 201 of the eighth sample preparation unit (Ac8).

The specimen 1 and the specimen 2 stored in the columns 201 of the seventh sample preparation unit (Ac7) and the eighth sample preparation unit (Ac8) are respectively aspirated by the pipette, and disposed in the waste bath 7.

The first sample preparation unit (Ac1) and the fifth sample preparation unit (Ac5) are used for activity measurement of the background, the third sample preparation unit (Ac3) and the fourth sample preparation unit (Ac4) are used for activity measurement of the CDK1, and the seventh sample preparation unit (Ac7) and the eighth sample preparation unit (Ac8) are used for activity measurement of the CDK2.

Therefore, by injecting the specimen remaining in the column into another column, the background activity measurement, the CDK1 activity measurement, and the CDK2 activity measurement can be performed with small amount of specimen.

The buffer 1 is then sent to the columns 201 to wash and remove unnecessary components in the specimen (step S33).

Subsequently, since the buffer 1 influences enzyme reaction executed in step S25, the buffer 2 is sent to the column 201 to wash off the components of the buffer 1 with the main aim of creating a condition for the relevant enzyme reaction (step S34).

The substrate reaction solution containing substrate Histon H1 and ATPγS is then injected to the column 201, and the piston 219 is reciprocated once (step S35). The liquid pushed out from the lower side of the column 201 is stored in the column 201 as it is. According to such step, the phosphate group is introduced to the Histon H1 with CDK1 and CDK2 as enzymes. The amount of phosphate group is influenced by the strength (i.e., activity value) of the work of the CDK1 or the CDK2 as enzyme, and thus the activity value of the CDK1 or the CDK2 can be obtained by measuring the amount of phosphate group. The background activity value obtained using the first sample preparation unit (Ac1) and the fifth sample preparation unit (Ac5) shown in FIG. 20 is used to perform background correction as hereinafter described.

The fluorescent labeled reagent is dispensed directly into the column 201 from above the column 201 using the pipette to bind the fluorescent label to the phosphate group introduced into the Histon H1 (step S36). In this case, the pipette repeats aspiration and discharge of liquid in the column for a predetermined time to stir the liquid in the column 201.

A reaction stopping solution is directly dispensed to the column 201 similar to the fluorescent labeled reagent after elapse of a predetermined time (e.g., for twenty minutes) from the start of step S26. The liquid in the column 201 is stirred by repeating aspiration and discharge of the liquid in the column for a predetermined time similar to step S26 (step S37). The binding of fluorescent label is thereby stopped.

The liquid in the columns 201 of the first sample preparation unit (Ac1), the third sample preparation unit (Ac3), the fourth sample preparation unit (Ac4), the fifth sample preparation unit (Ac5), the seventh sample preparation unit (Ac7), and the eighth sample preparation unit (Ac8) are dispensed to six wells of the solid phase tip for protein 101, and the solid phase tip for protein 101 is aspirated from the lower side (step S38). The Histon H1 containing phosphate group bound with fluorescent label is thereby solid-phased on the porous film of the phase tip for protein 101.

The well is washed similar to step S21 in the process of preparing the expression level measurement reagent (step S39).

Lastly, an operation of dispensing and discharging quenching reagent for quenching (background quenching) the fluorescent label that did not bind to the phosphate group introduced into the Histon H1 into wells is repeated six times (step S40).

(6) Analyzing Process

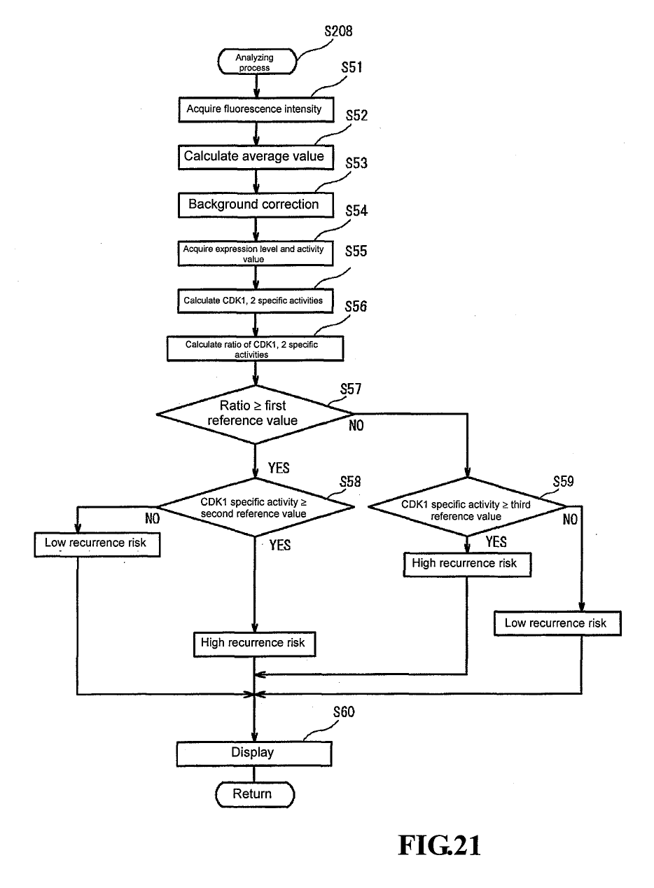

As shown in FIG. 21, in the step of analyzing process (step S208), analysis is performed from the fluorescence intensity obtained in the detecting unit, and the analysis result is output to the display 79.

First, the controller 77 acquires two fluorescent intensities for each of activity of CDK1, expression of CDK1, activity of CDK2, expression of CDK2, activity of background, and expression of background through the main body controller 10 from the light receiving system of the detecting unit 4 (step S51).

Thereafter, the controller 77 calculates the average value of the fluorescence intensities obtained two at a time for each item (step S52).

The background activity (average value) is subtracted from the fluorescence intensity (average value) of the CDK1 activity, and the background activity (average value) is subtracted from the fluorescence intensity (average value) of the CDK2 activity to perform background correction for the CDK1 activity and the CDK2 activity. The background correction is similarly performed for the CDK1 expression and the CDK2 expression (step S53).

Next, the expression level and the activity value are acquired using standard curve for each item (step S54). The standard curve is data for converting fluorescence intensity to expression amount or activity value, and is created in advance using two or more types of specimens which expression amount or activity value is known when the lot of the reagent is changed, and stored in the hard disc 91g of the controller 77.

The CDK1 specific activity and the CDK2 specific activity are calculated according to the following equation (step S55).

CDK1 specific activity=CDK1 activity value/CDK1 expression level

CDK2 specific activity=CDK2 activity value/CDK2 expression level

The ratio of the CDK1 specific activity and the CDK2 specific activity is calculated according to the following equation (Step S56).

Ratio of CDK1 specific activity and CDK2 specific activity=CDK2 specific activity/CDK1 specific activity

Determination is made on whether or not the ratio of the CDK1 specific activity and the CDK2 specific activity is greater than or equal to a first reference value (step S57). If Yes, the process proceeds to step S58, and determination on whether or not the CDK1 specific activity is greater than or equal to a second reference value, and if No, the process proceeds to step S59, determination is made on whether or not the CDK1 specific activity is greater than or equal to a third reference value.

In step S58, determination is made on whether or not the CDK1 specific activity is greater than or equal to the second reference value, and if Yes, the recurrence risk is determined to be “high”, and if No, the recurrence risk is determined to be “low”.

In step S59, determination is made on whether or not the CDK1 specific activity is greater than or equal to the third reference value, and if Yes, the recurrence risk is determined to be “high”, and if No, the recurrence risk is determined to be “low”.

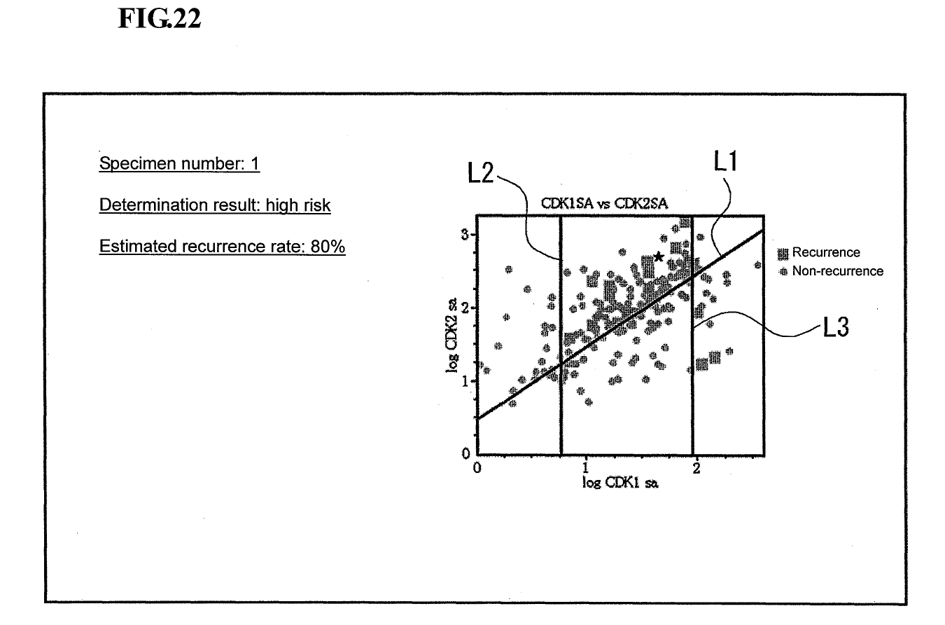

As shown in FIG. 22, the CDK1 specific activity and the CDK2 specific activity acting as the basis in determining the magnitude of the recurrence risk are plotted and displayed on the graph (displayed as * mark in the figure), and the determination result of the recurrence risk is displayed (step S60). The graph shown in FIG. 22 log displays the CDK1 specific activity on the horizontal axis and log displays the CDK2 specific activity on the vertical axis. The lines L1, L2, and L3 show first to third reference value, respectively. In the example shown in FIG. 22, the sample data used in determining the recurrence risk is thinly displayed for reference.

1) Reference Value Setting Method

The method of setting the first to the third reference values for determining “high” or “low” of the recurrence risk will be described.

[Storage of Sample Data]

First, the sample data in which measurement values of the predetermined items (e.g., CDK1 specific activity) of a great number of cancer patients and clinical information after the extirpation of the malignant tumor of the cancer patient are corresponded needs to be stored to obtain the reference values for acquiring the diagnosis support information of the cancer such as high and low of the possibility of cancer recurrence and high and low of the sensitivity (effectiveness) with respect to a specific anticancer drug. Such sample data is stored in the second database 91j of the hard disc 91g of the controller 77.

The predetermined items include each activity value of the first cyclin-dependent kinase (first CDK) and the second cyclin-dependent kinase (second CDK), each expression level of the first CDK and the second CDK, the ratio (first specific activity) of the activity value of the first CDK and the expression level of the first CDK, the ratio (second specific activity) of the activity value of the first CDK and the expression level of the second CDK, the ratio (specific activity ratio) of the first specific activity and the second specific activity, and the like, which are measured using the diagnosis support apparatus.

The clinical information is information containing the therapeutic regimen of the cancer patient extirpated with malignant tumor, postoperative progress, and the like, and can include information such as presence of lymph node metastasis, postoperative therapy (no therapy, hormonal therapy, chemotherapy, etc.), presence of recurrence, number of days from extirpation until recurrence, sensitivity (effectiveness) with respect to anticancer drug, survival rate, and the like.

FIGS. 25 and 26 show one part of the example of sample data collected from patients subjected to extirpative operation of breast cancer in a table. In the table, “No.” is a number such as specimen number and patient number for identifying the patient, and “Age” and “Mens” respectively indicate the age of patient and the before and after of menopause. Furthermore, “T” indicates the size of the extirpated tumor, wherein the unit is in “mm” if the indication is numeric characters, and “a” represents smaller than or equal to 2 cm, “b” represents 2 to 5 cm, and “c” represents greater than or equal to 5 cm if the indication is roman letters. Information related to the specimen such as collected amount of specimen to be measured and degree of blood mixture is appropriately included in the sample data.

In FIGS. 25 and 26, the portion indicated by M is the measurement item, and the portion indicated by C is the clinical information. The content of each measurement item is as follows.

CDK1A: activity value of CDK1

CDK2A: activity value of CDK2

CDK1E: expression level of CDK1

CDK2E: expression level of CDK2

CDK1SA: specific activity of CDK1

CDK2SA: specific activity of CDK2

CDK2/1: ratio of specific activity of CDK1 and specific activity of CDK2

The content of each of clinical information is as follows.

Postoperative aid: information of therapy performed after the extirpative operation, wherein postoperative aid 1 is hormonal therapy, and postoperative aid 2 is chemotherapy. In the case of hormonal therapy, type of hormone drug used in the treatment is described.

TAM: tamoxifen

TOR: toremifene

ZOL: zoladex

Aromacin; Aromatase Inhibitor

Arimidex; Aromatase Inhibitor

In the case of chemotherapy, the type of anticancer drug is described.

CMF: Cyclo-phosphamide, Methotrexate, Fluorouracil

CEF: Cyclo-phosphamide, Epirubicin (anthracyclin antibiotic), Fluorouracil

CE: Cyclo-phosphamide, Epirubicin

Wherein “0” indicates no implementation.

Recurrence: indicate presence of recurrence, wherein “1” indicates that recurrence is not present and “0” indicates that recurrence is present.

DFS: number of days from extirpative operation until recurrence.

Although not shown in FIG. 25, the sample data may also contain the following clinical information.

LN: presence of lymph node metastasis (0: no, 1: yes)

ER, PR: determination of effectiveness of hormonal therapy, wherein ER indicates presence of estrogen receptor, and PR indicates presence of progesterone receptor. “1” indicates presence and “0” indicates no presence.

HG: indicates pathological malignancy in three stages of one to three, wherein “3” is the worst.

HER2: expression level or number of genes of HER2.

Such sample data can be created in correspondence to various cancers such as gastric cancer and lung cancer in addition to breast cancer. The clinical information is appropriately selected according to the type of cancer.

[Setting of Reference Value]

The measurement item assumed to have correlation with the risk of recurrence of cancer is selected, and information related to such measurement item is set as index or parameter. For instance, when determined that there is a strong correlation between the recurrence risk of a certain type of cancer and the specific activity of the CDK1, and the risk of cancer recurrence becomes higher as the specific activity of the CDK1 becomes larger, the specific activity of the CDK1 is set as the index and a certain value of the relevant specific activity is set as a reference value, wherein the recurrence risk is “high” for cases greater than the relevant value and the recurrence risk is “low” for cases smaller than the relevant value. The reference value is selected such that “high” is set for the possibility of recurrence within five years after the operation of greater than or equal to 50% and “low” is set for less than 50%. The segmentation of the recurrence risk is not only two stages of “high” and “low” but may be three stages of “high”, “medium”, and “low”, or more, and is not particularly limited in the present invention.

The index correlated with the recurrence risk of the cancer is not limited to one and two or more may be selected. For instance, in addition to the specific activity of CDK1, the specific activity of CDK2, and the ratio (specific activity ratio) of the specific activity of the CDK1 and the specific activity of the CDK2, may be measurement items for setting the reference value. In the specification, “measurement item” is a concept including, in addition to the activity value and the expression level of the CDK which values are directly obtained by the measurement device, the specific activity calculated based on such activity value and the expression level, the specific activity ratio, and the like. In addition to the items related to the CDK, HER2 etc. may be included.

The relationship between the recurrence risk of the cancer and the measurement item can be shown in a graph using the sample data. The view shown on the upper right of FIG. 23 is a scatter graph created using the following information on patients without lymph node metastasis and only treated with hormonal therapy among the patients with breast cancer.

Recurrence: presence of recurrence

CDK1SA: specific activity of CDK1

CDK2SA: specific activity of CDK2

CDK2/1: ratio of specific activity of CDK1 and specific activity of CDK2

The vertical axis of the scatter graph is the log value (log value for the sake of easy understanding) of the specific activity of the CDK2, and the horizontal axis of the scatter graph is the log value of the specific activity of the CDK1. The following three are set as a reference value used in risk determination.

First reference value: CDK2/1 is 5.0

Second reference value: CDK1 specific activity is 6

Third reference value: CDK1 specific activity is 90

In the case of breast cancer, generally, cases where recurrence rate within five years after the operation is predicted to be lower than or equal to 5% are determined as having low recurrence risk, and similarly, cases where recurrence rate within five years after the operation is predicted to exceed 15% are determined as having high recurrence risk when subjected to only hormonal therapy, and thus the first to the third reference values are set so that cases that recurred within five years after the operation have the predetermined proportion. In the scatter graph shown in FIG. 23, no recurrence case is found when the specific activity of the CDK1 is smaller than a certain value, the recurrence cases increase as the ratio of the specific activity becomes larger than the certain value, and when the specific activity of the CDK1 becomes larger than the certain value, the recurrence cases are found irrespective of the value of the ratio of the specific activity, whereby the sample data is divided into six regions, and “high (H)” region and “low (L)” region are set as shown on the left lower of FIG. 23. The three reference values are set so that the proportion of recurrence within five years after the operation of the sample data contained in the “low” region becomes lower than or equal to 5%, and the proportion of recurrence within five years after the operation of the sample data contained in the “high” region exceeds 15% (about 20%). In the figure shown at the left lower of FIG. 23, a button is arranged in correspondence to each region, so that “high” or “low” can be set by clicking the button. The scatter graph shown in FIG. 23 can be created depending on the type of cancer, and can be created for every specific therapy (include no therapy). For instance, even with respect to the patient with the same breast cancer, a similar scatter graph having the specific activity of the CDK1, the specific activity of the CDK2, and the ratio of the specific activity of the CDK1 and the specific activity of the CDK2 as indices can be created with respect to a patient group subjected to a specific chemotherapy.

(9) Overall Flow of Process by the Diagnosis Support Apparatus A (Second Embodiment)

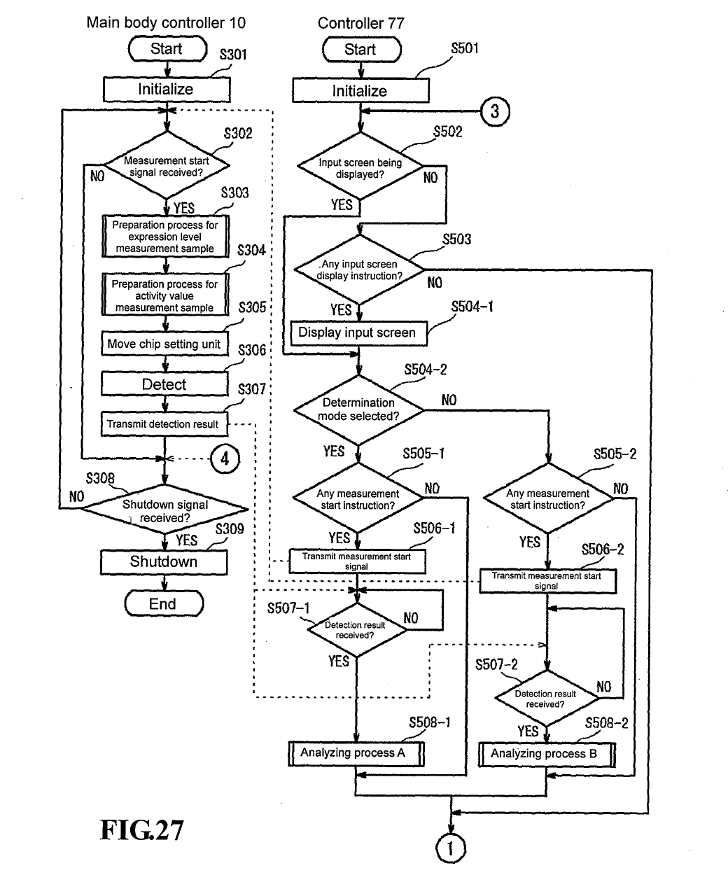

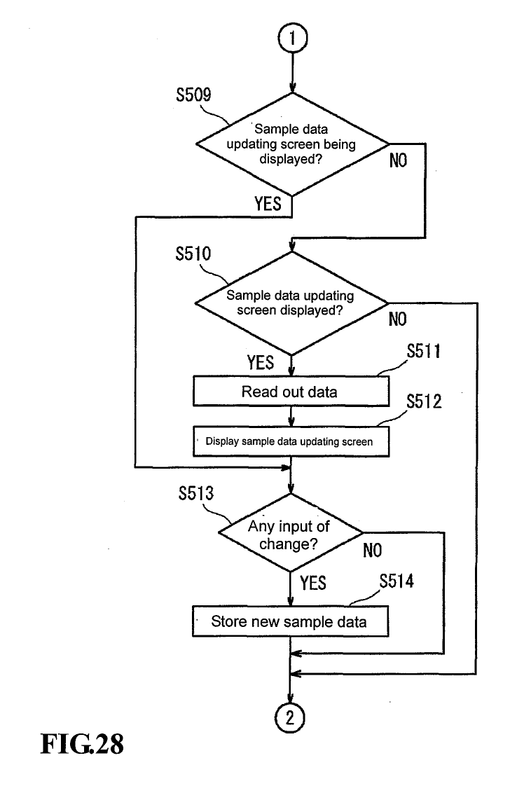

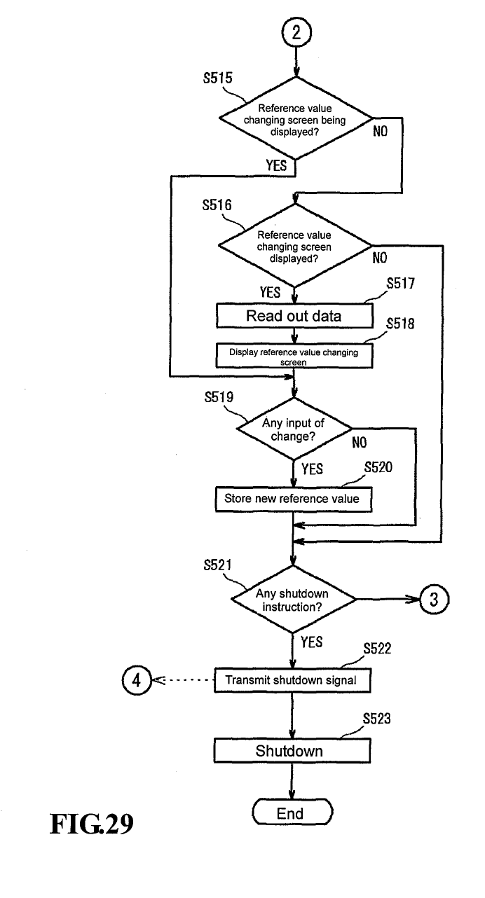

The overall flow of the process according to the second embodiment by the diagnosis support apparatus A is shown in FIGS. 27 to 29. The process according to the second embodiment mainly differs from the process of the first embodiment in including a step in which the user selects either a determination mode for simply performing measurement and analysis, and a sample data updating mode of storing the results of measurement and analysis in the storage member of the device as new sample data after accepting the input of information such as specimen number.

When the power of the apparatus body 20 is turned ON, initialization of the main body controller 10 is performed (step S301). In this initialization operation, initialization of the program, return to an origin position for the driving member of the apparatus body 20, and the like are performed.

When the power of the personal computer 12 is turned ON, initialization of the controller 77 is performed (step S501). In this initialization operation, initialization of the program or the like is performed. After the initialization is completed, a menu screen (not shown) including an input screen display button for instructing the display of an input screen is displayed on the display 79. The user can operate the input unit 78 to select the input screen button for instructing the display of the input screen of the menu screen.

In step S502, the controller 77 determines whether or not the input screen is being displayed. The controller 77 advances the process to step S504-2 if determined that the input screen is being displayed (Yes), and advances the process to step S503 if determined that the input screen is not being displayed (No).

In step S503, the controller 77 determines whether or not a display instruction of the input screen has been made (whether or not input screen button for instructing the display of the input screen of the menu screen is selected). The controller 77 advances the process to step S504-1 if determined that the display instruction of the input screen has been made (Yes), and advances the process to step S509 if determined that the display instruction of the input screen has not been made (No).

In step S504-1, the controller 77 displays the input screen on the display 79. The user operates the input unit 78 to input information related to the measurement such as specimen number. Furthermore, the user operates the input unit 78 to select either the determination mode (mode of simply performing measurement and analysis) or the sample data updating mode (mode of storing the results of measurement and analysis in the storage member of the device as new sample data). Specifically, the input button of the two modes is displayed on the display 79 of the personal computer 12. The operator (user) selects the input button of the desiring mode by operating the input unit 78. The user selects the start button displayed on the input screen by operating the input unit 78 to give the instruction to start the measurement.

The user selects the start button displayed on the input screen by operating the input unit 78 to give the instruction to start the measurement.

In step S504-2, the controller 77 determines whether or not the determination mode is selected. The controller 77 advances the process to step S505-1 if determined that the determination mode is selected (Yes), and advances the process to S505-2 if determined that the determination mode is not selected (No).

If determined that the determination mode is selected in step S504-2, the controller 77 determines whether or not the instruction to start the measurement is made in step S505-1. The controller 77 advances the process to step S506-1 if determined that the instruction to start the measurement is made (Yes), and advances the process to step S509 if determined that the instruction to start the measurement is not made (No). In step S506-1, a measurement start signal is transmitted from the controller 77 to the main body controller 10.

In step S302, the main body controller 10 determines whether or not the measurement start signal is received. The main body controller 10 advances the process to step S303 if determined that the measurement start signal is received (Yes), and advances the process to step S308 if determined that the measurement start signal is not received (No).

In step S303, the sample for expression level measurement is prepared. The specimen is aspirated from the specimen container set in the first reagent setting unit 5. A predetermined process is performed on the aspirated specimen, and the sample for expression level measurement is prepared.

In step S304, the sample for activity value measurement is prepared. The specimen is aspirated from the specimen container set in the first reagent setting unit 5. A predetermined process is performed on the aspirated specimen, and the sample for activity value measurement is prepared.

The details of preparation of the expression level measurement sample in step S303 and the preparation of the activity value measurement sample in step S304 are the same as step S3 (FIG. 18) and step S4 (FIG. 19) of the first embodiment, respectively, and thus the description thereof will be omitted.

In step S305, the chip setting unit 1 set with the solid phase tip for protein 101 including the sample for expression level measurement and the sample for activity value measurement moves into the detecting unit 4 from the position shown in FIG. 1.

In step S306, excitation light is irradiated on each well of the solid phase tip for protein 101, and fluorescence radiated from each sample is detected.

In step S307, the detected detection result is transmitted from the main body controller 10 to the controller 77.

In step S507-1, the controller 77 determines whether or not the detection result is received. The controller 77 advances the process to step S508-1 if determined that the detection result is received (Yes). The controller 77 again executes the process of step S507-1 if determined that the detection result is not received (No).

In step S508-1, the controller 77 executes an analyzing process (analyzing process A) from the acquired detection result, and outputs the analysis result.

If determined that the determination mode is not selected in step S504-2 since the sample data storage mode is selected, the controller 77 determines whether or not instruction to start the measurement is made in step S505-2. The processes from step S505-2 to step S507-2 are the same as from step S505-1 to step S507-1, and thus the detailed description thereof will be omitted.

In step S508-2, the controller 77 executes an analyzing process (analyzing process B) from the acquired detection result, and outputs the analysis result.

In step S509, the controller 77 determines whether or not a sample data updating screen is displayed. The controller 77 advances the process to step S513 if determined that the sample data updating screen is displayed (Yes), and advances the process to step S510 if determined that the sample data updating screen is not displayed (No).

In step S510, the controller 77 determines whether or not the display instruction of the sample data updating screen has been made. The controller 77 advances the process to step S511 if determined that the display instruction of the sample data updating screen has been made (Yes), and advances the process to step S515 if determined that the display instruction of the sample data updating screen has not been made (No).

In step S511, the RAM 91c of the controller 77 reads out the sample data stored in the second database 91j of the hard disc 91g.

In step S512, the sample data updating screen including the read sample data is displayed on the display 79. The user operates the input unit 78 to select the item to be changed, and information after change is input for the selected item, similar to the first embodiment.

In step S513, the controller 77 determines whether or not input of new sample data has been made. The controller 77 advances the process to step S514 if determined that input of the new sample data has been made (Yes), and advances the process to step S515 if determined that input of the new sample data has not been made (No).

In step S514, the input new sample data is stored in the second database 91j of the hard disc 91g.

In step S515, the controller 77 determines whether or not a reference value updating screen is being displayed. The controller 77 advances the process to step S519 if determined that the reference value updating screen is being displayed, and advances the process to step S516 if determined that the reference value updating screen is not being displayed (No).

In step S516, the controller 77 determines whether or not a display instruction of the reference value updating screen has been made. The controller 77 advances the process to step S517 if determined that the display instruction of the reference value updating screen has been made (Yes), and advances the process to step S521 if determined that the display instruction of the reference value updating screen has not been made (No).

In step S517, the RAM 91c of the controller 77 reads out the sample data stored in the second database 91j and the reference value stored in the first database 91i of the hard disc 91g

In step S518, the reference value updating screen (see FIGS. 23 and 24 described in relation to the first embodiment) including a graph created based on the read sample data and reference value is displayed on the display 79.

In step S519, the controller 77 determines whether or not an input to change the reference value has been made. The controller 77 advances the process to step S520 if determined that an input of the new reference value has been made (Yes), and advances the process to step S521 if determined that an input of the new reference value has not been made (No).

In step S520, the input new reference value is stored in the first database 91i of the hard disc 91g.

In step S521, the controller 77 determines whether or not an instruction to shutdown is accepted. The controller 77 advances the process to step S522 if determined that the instruction to shutdown is accepted (Yes), and returns the process to step S502 if determined that the instruction to shutdown is not accepted (No). In step S522, a shutdown signal is transmitted from the controller 77 to the main body controller 10. In step S523, the controller 77 performs the process of shutting down the personal computer 12, and completes the process.

In step S308, the main body controller 10 determines whether or not the shutdown signal has been received. The main body controller 10 advances the process to step S309 if determined that the shutdown signal has been received (Yes), and returns the process to step S302 if determined that the shutdown signal has not been received (No). In step S309, the main body controller 10 shuts down the apparatus body 20, and terminates the process.

(10) Analyzing Process A (Step S508-1)

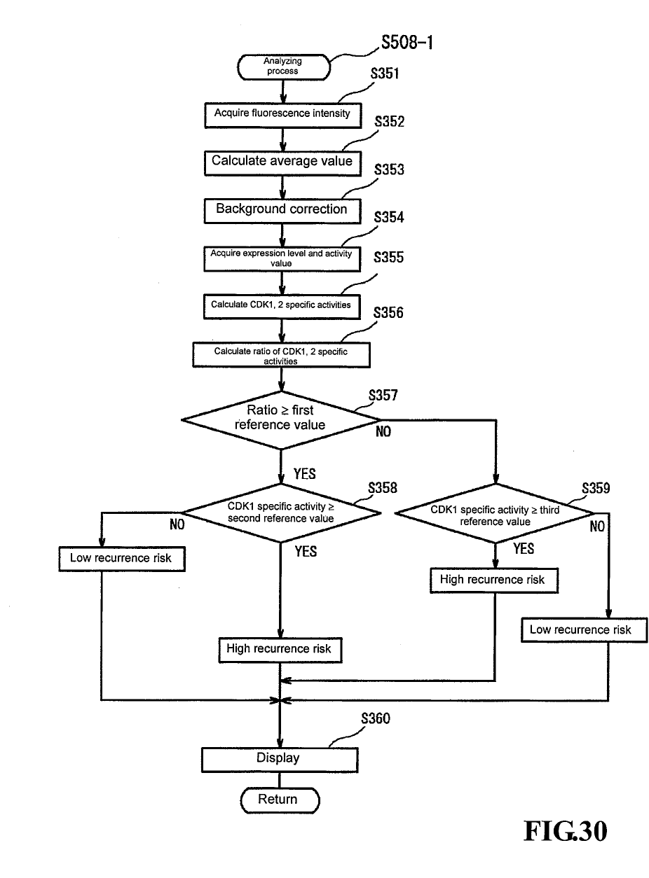

As shown in FIG. 30, analysis is performed from the fluorescence intensity obtained in the detecting unit, and the analysis result thereof is output. This process is similar to the analyzing process shown in FIG. 21.

First, the controller 77 acquires two fluorescent intensities for each of activity of CDK1, expression of CDK1, activity of CDK2, expression of CDK2, activity of background, and expression of background through the main body controller 10 from the light receiving system of the detecting unit 4 (step S351).

Thereafter, the controller 77 calculates the average value of the fluorescence intensities obtained two at a time for each item (step S352).

The background activity (average value) is subtracted from the fluorescence intensity (average value) of the CDK1 activity, and the background activity (average value) is subtracted from the fluorescence intensity (average value) of the CDK2 activity to perform background correction for the CDK1 expression and the CDK2 expression (step S353).

Next, the expression level and the activity value are acquired using standard curve for each item (step S354). The standard curve is data for converting the fluorescence intensity to expression amount or activity value, and is created in advance using two or more types of specimens which expression amount or activity value is known when the lot of the reagent is changed, and stored in the hard disc 91g of the controller 77.

The CDK1 specific activity and the CDK2 specific activity are calculated according to the following equation (step S355).

CDK1 specific activity=CDK1 activity value/CDK1 expression level

CDK2 specific activity=CDK2 activity value/CDK2 expression level

The ratio of the CDK1 specific activity and the CDK2 specific activity is calculated according to the following equation (Step S356).

Ratio of CDK1 specific activity and CDK2 specific activity=CDK2 specific activity/CDK1 specific activity

Determination is made on whether or not the ratio of the CDK1 specific activity and the CDK2 specific activity is greater than or equal to a first reference value (step S357). If Yes, the process proceeds to step S358, and determination on whether or not the CDK1 specific activity is greater than or equal to a second reference value, and if No, the process proceeds to step S359, determination is made on whether or not the CDK1 specific activity is greater than or equal to a third reference value.

In step S358, determination is made on whether or not the CDK1 specific activity is greater than or equal to the second reference value, and if Yes, the recurrence risk is determined to be “high”, and if No, the recurrence risk is determined to be “low”.

In step S359, determination is made on whether or not the CDK1 specific activity is greater than or equal to the third reference value, and if Yes, the recurrence risk is determined to be “high”, and if No, the recurrence risk is determined to be “low”.

As shown in FIG. 22, the CDK1 specific activity and the CDK2 specific activity acting as the basis in determining the magnitude of the recurrence risk are plotted and displayed on the graph (displayed as * mark in the figure), and the determination result of the recurrence risk is displayed (step S360). The graph shown in FIG. 22 log displays the CDK1 specific activity on the horizontal axis and log displays the CDK2 specific activity on the vertical axis. The lines L1, L2, and L3 show first to third reference value, respectively. In the example shown in FIG. 22, the sample data used in determining the recurrence risk is thinly displayed for reference.

(11) Analyzing Process B (Step S508-2)

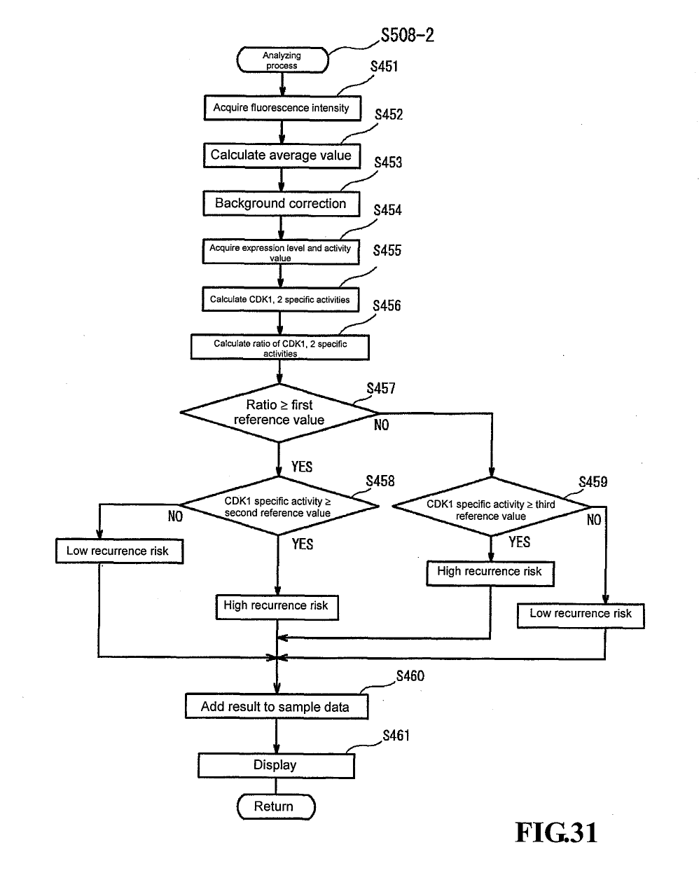

As shown in FIG. 31, analysis is obtained from the fluorescence intensity obtained in the detecting unit, and the analysis result thereof is output. In the analyzing process B, steps from step S451 of acquiring the fluorescence intensity to step S459 of comparing the specific activity of the CDK1 and the third reference value are exactly the same as steps S351 to 359 in the analyzing process A, and thus the description thereof will be omitted.

In the analyzing process B, the measurement value and the analysis result (include determination result) of specimen are added to the sample data, that is, stored in the hard disc 91g of the controller 77 (step S460) after the determination of the recurrence risk is performed.

As shown in FIG. 22, the CDK1 specific activity and the CDK2 specific activity acting as the basis in determining the magnitude of the recurrence risk are plotted and displayed on the graph (displayed as * mark in the figure), and the determination result of the recurrence risk is displayed (step S461).

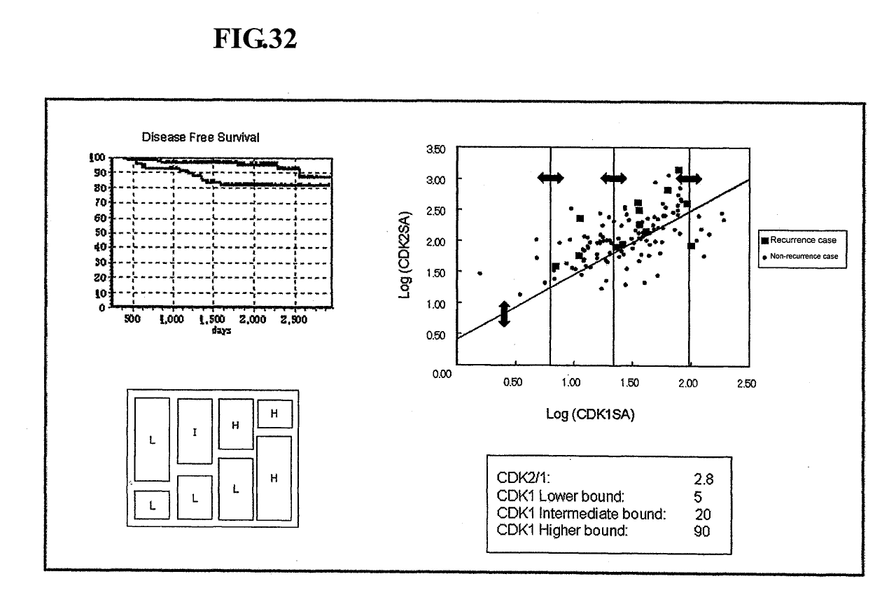

In the first and the second embodiments, the recurrence risk is determined as “high” or “low”, but determination may not be two stages and may be three or more stages. FIG. 32 shows an example of a reference value changing screen with three determination regions of “high”, “medium”, and “low”. This example is the same as the example shown in FIG. 23 in that the specific activity of CDK1 and the ratio (specific activity ratio) between the specific activity of CDK1 and the specific activity of CDK2 are used as indices for recurrence risk determination. However, in the example shown in FIG. 32, the medium reference value (=20) is set in addition to the low reference value (=5) and the high reference value (=90) for the specific activity of CDK1, and four references including the specific activity ratio are used.

The eight regions segmented by the four reference values are set to one of “high” recurrence risk, “medium” recurrence risk, or “low” recurrence risk. Specifically, the region larger than the reference value related to the specific activity ratio and larger than the medium reference value is the “high” region. The region larger than the reference value related to the specific activity ratio, smaller than the medium reference value, and larger than the low reference value is the “medium” region. Furthermore, the region larger than the reference value related to the specific activity ratio and smaller than the low reference value is the “low” region.

For regions smaller than the reference value related to the specific activity ratio, the region larger than the high reference value is “high” region, and the region smaller than the high reference value is “low” region.

In this example as well, the reference value can be changed by placing the pointer on a linear line representing the reference value in the graph and dragging the pointer, or arranging a scroll bar or a button (not shown) in the screen, and operating the same. In the example shown in FIG. 32, three types of survival curves corresponding to high, medium, and low risk are crated and displayed. In this example as well, when the reference value is changed, a survival curve is newly created based on the sample data contained in the region segmented by the reference value after change, and then displayed. Therefore, the user can appropriately change the reference value while looking at the survival curve so that the recurrence rate after elapse of five years takes a value within a predetermined range, and as a result, can set a reference value of high determination accuracy.

In the embodiment described above, the value calculated from the activity values and the expression levels of CDK1 and CKD2 is used as a index for recurrence risk determination, but the present invention is not limited thereto, and an index different from the value calculated from the activity values and the expression levels of CDK1 and CKD2 can be used, and furthermore, the value calculated from the activity values and the expression levels of CDK1 and CKD2 and another index may be used in combination.

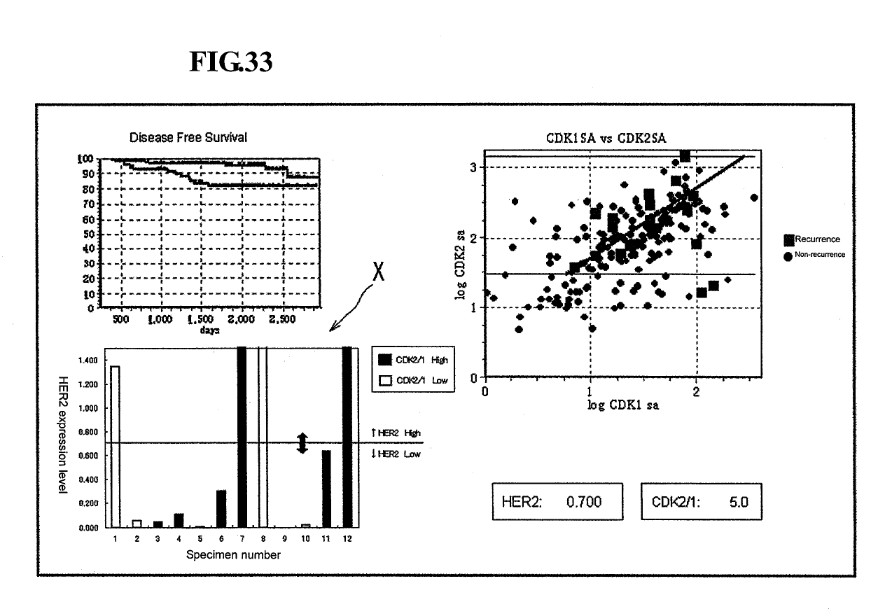

FIG. 33 shows an example of a reference value changing screen of the case of determining the recurrence risk by combining the ratio (specific activity ratio) between the specific activity of CDK1 and the specific activity of CDK2, and the expression level of the HER2.

In this example, the reference value of the expression level of HER2 is set to 0.7, wherein “High” is the case when the expression level of HER2 is greater than or equal to 0.7, and “Low” is for when the expression level of HER2 is less than 0.7. The reference value of the specific activity ratio (CDK2/1) is set to 5.0, wherein “High” is the case when the CDK2/1 is greater than or equal to 5.0, and “Low” is the case when the CDK2/1 is less than 5.0.

In FIG. 33, the graph shown with X indicates the HER2 expression level of each patient specimen, and the specimen number painted black indicates that CDK2/1 is “High”.

In this example, high, medium, and low of the recurrence risk is determined in the following manner. If both the specific activity ratio and the HER2 expression level are “Low”, the recurrence risk is determined as “Low”. If both the specific activity ratio and the HER2 expression level are “High”, the recurrence risk is determined as “High”. If one is “High” and the other is “Low”, the recurrence risk is determined as “Medium”.

In the example shown in FIG. 33 as well, the reference value can be changed with a method similar to the example shown in the first and second embodiments and FIG. 32, and a survival curve is newly created by changing the reference value, and then displayed.

In the embodiment described above, the diagnosis support apparatus for determining the malignancy (recurrence risk) of cancer is described by way of example, but the present invention is not limited thereto. The present invention is also applicable to the diagnosis support apparatus for determining the effectiveness (sensitivity) of the anticancer drug.

A method of predicting the sensitivity of the anticancer drug using CDK will be described.

The method of predicting the sensitivity of the anticancer drug includes a step of comparing at least one parameter selected from a group consisting of activity value and expression level of a cyclin-dependent kinase of a malignant tumor cell collected from a patient, and a ratio between the activity value and the expression level, with a reference value corresponding to the selected parameter, and a step of predicting the sensitivity of the anticancer drug of the patient based on the result of the comparing step.

If predicted that the sensitivity of the anticancer drug is high in the prediction method, it is considered that there is a high possibility of the source of origin shrinking or disappearing as a result of administering the anticancer agent while the source of origin exists if before operation. If after the operation, it is considered that there is a high possibility the cancer will not recur by administering the anticancer drug after performing the extirpative operation of the tumor.

On the other hand, if predicted that the sensitivity of the anticancer drug is low in the prediction method, it is considered that there is a low possibility of the source of origin shrinking or disappearing as a result of administering the anticancer agent while the source of origin exists if before operation. If after the operation, it is considered that there is a high possibility the cancer will recur even if the anticancer drug is continuously administered after performing the extirpative operation of the tumor.

The tissue that serves as a sample used in the prediction method is a tissue that contains the malignant tumor cell collected from the patient. In the case of postoperative therapy, the tissue can be collected through extirpative operation of the cancer, and such tissue can be used. In the case of preoperative therapy, biopsied tissue (biopsy) from the tumor tissue of the patient is used.

The cyclin-dependent kinase (CDK) used in the prediction method includes CDK1, CDK2, CDK4, CDK6, cyclin A-dependent kinase, cyclin B-dependent kinase, cyclin D-dependent kinase, and cyclin E-dependent kinase, and is appropriately selected depending on the type of cancer and type of anticancer drug. That is, there are various types of cancers, and the nature related to the cell cycle of the cancerous cell of each patient is greatly related to the sensitivity of the anticancer drug.

The types of cancer include breast cancer, gastric cancer, large intestine cancer, esophageal cancer, ovarian cancer, prostate cancer, and the like. The anticancer drug includes, for breast cancer, CMF group (therapy of simultaneously administering three drugs of cyclo-phosphamide, methotrexate, and fluorouracil), taxane anticancer drug such as docetaxel and paclitaxel, CE (therapy of simultaneously administering two drugs of cyclo-phosphamide, and epirubicin), AC (therapy of simultaneously administering two drugs of doxorubicin and cyclophosphamide), CAF (therapy of simultaneously administering three drugs of fluorouracil, doxorubicin, and cyclophosphamide), FEC (therapy of simultaneously administering three drugs of fluorouracil, epirubicin, and cyclophosphamide), a therapy of simultaneously administering two drugs of Trastuzumab and paclitaxel, capecitabine, and the like; for gastric cancer, FAM (therapy of simultaneously administering three drugs of fluorouracil, doxorubicin, and mitomycin), FAP (therapy of simultaneously administering three drugs of fluorouracil, doxorubicin, and cisplatin), ECF (therapy of simultaneously administering three drugs of epirubicin, cisplatin, and fluorouracil), a therapy of simultaneously administering two drugs of mitomycin C and tegafur, a therapy of simultaneously administering two drugs of fluorouracil and carmustine, and the like; for large intestine cancer, a therapy of simultaneously administering two drugs of fluorouracil and leucovorin, a therapy of simultaneously administering two drugs of mitomycin and fluorouracil, and the like; and for ovarian cancer, TP (therapy of simultaneously administering two drugs of paclitaxel and cisplatin), TJ (therapy of simultaneously administering two drugs of paclitaxel and carboplatin), CP (therapy of simultaneously administering two drugs of cyclophosphamide and cisplatin), CJ (therapy of simultaneously administering two drugs of cyclophosphamide and carboplatin), and the like.

The index used in predicting the sensitivity of the anticancer drug is one or two parameters selected from the activity value and the expression level of the CDK, and the ratio between the activity value and the expression level. The ratio between the activity value and the expression level may be a CDK specific activity expressed as CDK activity value/CDK expression level, or a value expressed as CDK expression level/CDK activity value (inverse of CDK specific activity).

The sensitivity with respect to the anticancer drug of the cell can be predicted by comparing the parameter with the predetermined reference value. The parameter selected from the activity value, the expression level, and the ratio between the activity value and the expression level is a parameter appropriately selected depending on the type of anticancer agent and type of cancer. Regarding this parameter, for the tumor cell extirpated and saved before the anticancer drug treatment from the cancer patient subjected to anticancer drug treatment in the past and which result is known, the activity value and the expression level of the CDK are measured, and the anticancer drug treatment result is analyzed for each parameter to have the parameter having correlation with the anticancer drug treatment result as the parameter to be used in predicting the sensitivity of the anticancer drug.

The parameter to be compared with the reference value may be one parameter in a predetermined CDK or a combination of two parameters. If two parameters are selected, each parameter is compared with the respective reference value.

The CDK that serves as the index of determination may be one type (first sensitivity prediction method) or two or more types (second sensitivity prediction method).

When using two or more types of CDK, the parameter in each of the plurality of CDKs and the respective reference values are compared, and the effectiveness is predicted by the combination of the comparison result of each kinase (second (1) sensitivity prediction method). In this case, the type of parameter to be compared with the reference value may be the same type of parameter (e.g., expression level) for the plurality of CDKs, or different types of parameters (e.g., compare expression level for one CDK, and compare activity value for the other CDK).

When using a plurality of types of CDKs, the first sensitivity prediction method is predicted based on the first CDK, and for tumor cells which sensitivity is predicted as low in the first sensitivity prediction method, the parameter selected from the activity value, the expression level, and the ratio between the activity value and the expression level and the reference value corresponding to the parameter are compared to predict the sensitivity for the CDK different from the first CDK (second (2) sensitivity prediction method). In the second (2) sensitivity prediction method, the CDK different from the first CDK may be one type of CDK (second CDK) or a plurality of types of CDKs (third, fourth, . . . CDK). When using a plurality of types of CDKs, at least one parameter selected from a group consisting of the activity value, the expression level, and the ratio between the activity value and the expression level of each CDK and the reference value corresponding to the parameter are compared for each CDK, and the sensitivity of the anticancer drug of the patient is predicted based on the combination of such comparison results.

Regarding the second CDK, the parameter used to predict the sensitivity is selected from the above group. Only one parameter may be selected or two parameters may be selected, and compared with the respective reference value. When measuring for a plurality of types of CDKs as different CDKS, that is, for second, third, and fourth CDK, the parameter used in the determination may all be the same type of parameter (e.g., expression level), or may be different types of parameters (e.g., combination of using expression level for the second CDK, and activity value for the third CDK).

The second sensitivity prediction method is effective in raising the accuracy rate of the prediction. Furthermore, according to the second (2) sensitivity prediction method, cases where anticancer drug effectively operate are not few even if predicted that the sensitivity is low in the first sensitivity prediction method, and thus is meaningful to use the second sensitivity prediction method.

The efficacy of the anticancer drug can be classified to a level of preventing the disease from worsening and a level of shrinking the tumor and curing the disease, where prediction taking into view the level of efficacy of the anticancer drug is performed according to the second sensitivity prediction method, in particular, second (2) prediction method.

The prediction method may compare not only the CDK, but the expression level of the cyclin-dependent kinase inhibitor (CDK inhibitor) with the corresponding reference value, and predict the sensitivity of the anticancer drug of the patient based on the combination of the comparison result of the CDK and the comparison result of the CDK inhibitor (third sensitivity prediction method). The CDK inhibitor is a factor group that bonds with the cyclin CDK complex and inhibits the activity, and is classified into the INK4 family and the CIP/KIP family. In the method of predicting the sensitivity, CIP/KIP is preferably used, in particular, p21 is preferably used. P21 is an inhibitor that inhibits the progression at both G1 period and G2 period checkpoint in the cell growth cycle, thereby creating a time to repair the damaged DNA.

The third sensitivity prediction method may predict the sensitivity of the anticancer drug based on the combination of the result of comparing the CKD with the reference value for a predetermined parameter and the result of comparing the expression level of the CDK inhibitor with the reference value; or comparing the CDK with the reference value for a predetermined parameter in the first stage and prediction determining the sensitivity (first sensitivity prediction method), and thereafter comparing the expression level of the CDK inhibitor with the reference value in the second stage for the tumor cell predicted to have low sensitivity and selecting that with high sensitivity.

HER2 and p21 are reported as a treatment effectiveness prediction factor of the CMF administration group. In the trial of the International Breast Cancer Study Group (IBCSG), the CMF administration is found to be invalid for breast cancer patients where HER2 is expressed in excess, and with respect to p21, the non-disease survival rate in the p21 high expression patient group is found to be significantly worse than the low expression patient group. However, both HER2 and p21 are factors for predicting the patient group having low effect to CMF therapy, and no reports have been made on the factors for actively predicting the patient group with treatment effect. The prediction method actively indicates that the treatment of the anticancer drug is effective, and presents cases where effectiveness close to 100% can be expected by making the reference value stricter.

In the prediction method, the reference value is a value appropriately set depending on the type of anticancer drug and the type of cancer. Specifically, the relationship between the anticancer drug treatment result of administering a predetermined anticancer drug with respect to a predefined cancer patient and the parameter is examined for numerous anticancer drug treatment results, and the value is set so as to be able to select a case where the anticancer drug treatment result is effective with respect to the parameter having correlation with a great number of anticancer drug treatment results. The reference value is preferably set so as to be able to select only a case where the anticancer drug treatment results are all effective. The result of anticancer drug treatment in a cancer patient reflects the sensitivity on the anticancer drug of the tumor cell of the relevant cancer patient. Thus, when the anticancer drug treatment result is effective for the cancer patient, the sensitivity on the anticancer drug of the tumor cell of the cancer patient is assumed as high. Thus, prediction of sensitivity with high accuracy can be made since the reference value is set based on the actual clinical treatment result. The accuracy of sensitivity prediction can be enhanced by increasing the number of clinical treatment results used in the setting of the reference value. The anticancer drug treatment result includes result of examining the change in tumor size when a predetermined anticancer drug treatment is continued and the presence of recurrence after continuing anticancer drug administration for five to six years.

The CDK specific activity calculated by CDK activity value/CDK expression value corresponds to the proportion of the CDK indicating activity of the CDK existing in the cell, and is the CDK activity level based on the growth state of the malignant tumor cell to be measured. The CDK specific activity obtained in this manner does not depend on the method of preparing the sample for the measurement. The cell solubilizing solution prepared from the biopsy material, in particular, the measurement sample preparation method, is likely to be influenced by the non-cellular tissue such as size of extracellular matrix contained in the actually collected tissue. Therefore, the unavoidable influence can be deduced in time of measurement sample preparation by using the CDK specific activity or the inverse number thereof, and sensitivity can be predicted at high accuracy even with the determination method focusing on protein.

The CDK inhibitor expression level is the target CDK inhibitor quantity (unit corresponding to number of molecules) measured from the cell solubilizing solution, and can be measured with a conventionally known method of measuring the target protein quantity from the protein mixture. ELISA method and western blot method may be used. The target protein (CDK inhibitor) may be trapped using a unique antibody. It may be a monoclonal antibody or a polyclonal antibody as long as it uniquely bonds with the target protein. In a case of trapping p21, both anti-p21 monoclonal antibody and anti-p21 polyclonal antibody can be used.

The diagnosis support apparatus for determining the sensitivity of the anticancer drug can be configured to determine the sensitivity of the anticancer drug using the first sensitivity prediction method described above, or can be configured to determine the sensitivity of the anticancer drug using the second sensitivity prediction method or the third sensitivity prediction method. Here, a configuration of changing the reference value used in the sensitivity determination of the anticancer drug using, by way of example, a diagnosis support apparatus for determining the sensitivity of taxane using CDK21 and p21 as the third sensitivity prediction method. In such diagnosis support apparatus, the specific activity of the CDK2 and the expression level of the p21 are acquired by analyzing the sample. The acquired specific activity of the CDK2 and the expression level of the p21 are compared with a predetermined reference value, whereby the sensitivity on taxane of the patient can be determined. In such diagnosis support apparatus, the reference value of the CDK2 and the reference value of p21 used in taxane sensitivity determination are stored in the storage member of hard disc and the like. The reference value of the CDK2 and the reference value of p21 can be changed on the screen.

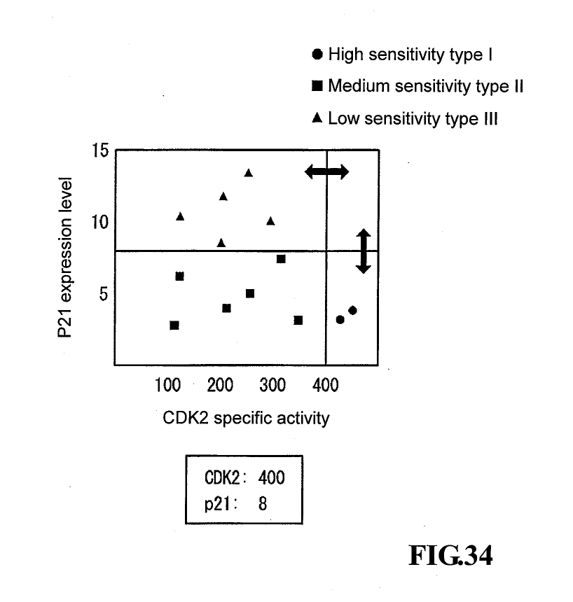

FIG. 34 shows an example of the reference value changing screen of the case of predicting the sensitivity of taxane or an anticancer drug with a combination of the specific activity of the CDK2 and the expression level of the p21. In this example, the reference value of the specific activity of the CDK2 is set to 400, wherein “high” is the case when the specific activity of the CDK2 is greater than or equal to 400, and “low” is the case when less than 400. The reference value of the expression level of p21 is set to 8, wherein “high” is the case when the expression level of p21 is greater than or equal to 400, and “low” is the case when less than 8.

The graph shown in FIG. 34 shows a scatter graph taking the expression level of p21 on the vertical axis, and the specific activity of the CDK2 on the horizontal axis. In the figure, data obtained from the patient of high taxane sensitivity type I is shown with a circle, data obtained from the patient of medium taxane sensitivity II is shown with a square, and data obtained from the patient of low taxane sensitivity III is shown with a triangle.

In this example, high, medium, and low of the recurrence risk is determined in the following manner. If the specific activity of CDK” is “high”, determination is made as high sensitivity type I. If the specific activity of the CDK2 is “low” and the expression level of the p21 is “high”, determination is made as low sensitivity type III. Furthermore, if both the specific activity of the CDK2 and the expression level of the p21 are “low”, determination is mad as medium sensitivity type II.

In the example shown in FIG. 34 as well, the reference value is changed by placing the pointer on a linear line representing the reference value in the graph and dragging the pointer, or arranging a scroll bar or a button (not shown) in the screen, and operating the same.

What is claimed is:

1. An apparatus for supporting diagnosis of cancer, comprising:
a memory for storing a predetermined reference value;
a measurement value acquirer for acquiring a measurement value of a predetermined item from a malignant tumor collected from a cancer patient;
a diagnosis support information preparer for preparing the diagnosis support information of cancer by comparing the acquired measurement value and the reference value; and
a change acceptor for accepting change of the reference value and for storing the changed reference value in the memory.

2. The apparatus of claim 1, wherein the diagnosis support information preparer prepares the diagnosis support information by comparing the acquired measurement value and the changed reference value after the changed reference value has been stored in the memory.

3. The apparatus of claim 1, comprising a display and a display controller for displaying the obtained diagnosis support information on the display.

4. The apparatus of claim 3, wherein the display controller displays a reference value changing screen for inputting the change of the reference value on the display, and the change acceptor accepts the input change of the reference value.

5. The apparatus of claim 4, wherein the display controller displays a graph representing a disease free survival on the reference value changing screen.

6. The apparatus of claim 3, wherein the memory stores a plurality of sample data, each of the sample data has a measurement value of the predetermined item of a cancer patient and clinical information of the cancer patient after extirpation of the malignant tumor, and the measurement value and the clinical information are associated with each other.

7. The apparatus of claim 6, wherein the display controller displays a screen including a sample data distribution chart based on the plurality of measurement values and the plurality of clinical information stored in the memory.

8. The apparatus of claim 7, wherein the display controller displays the screen including the sample data distribution chart which displays the reference value as a line movable on the sample data distribution chart; and
the change acceptor accepts change of the reference value by accepting movement of the line of the reference value on the sample data distribution chart.

9. The apparatus of claim 6, further comprising a data change acceptor for accepting change of the clinical information of the sample data stored in the memory, wherein the display controller displays a sample data updating screen for inputting the change of the clinical information on the display, and the data change acceptor accepts the input change of the clinical information.

10. The apparatus of claim 6, further comprising a data adder for adding sample data of a cancer patient to the memory.

11. The apparatus of claim 1, wherein the clinical information is at least one selected from a group consisting of presence or absence of recurrence, anticancer drug sensitivity, and survival rate.

12. The apparatus of claim 1, wherein the predetermined item includes item related to an expression and/or item related to activity of a cell cycle protein contained in the malignant tumor.

13. The apparatus of claim 12, wherein the predetermined item includes a specific activity of the cell cycle protein.

14. The apparatus of claim 1, wherein the diagnosis support information of the cancer is anticancer drug sensitivity or recurrence risk after extirpation of the malignant tumor of the cancer patient.

15. An apparatus for supporting diagnosis of cancer, comprising:
a memory for storing a predetermined reference value;
a measuring unit for measuring a malignant tumor collected from a cancer patient;
a measurement value obtainer for obtaining a measurement value of a predetermined item from the malignant tumor;
a diagnosis support information preparer for preparing the diagnosis support information of cancer by comparing the acquired measurement value and the reference value;
a display;
a display controller for displaying the obtained diagnosis support information on the display; and
a change acceptor for accepting change of the reference value and for storing the changed reference value in the memory.

16. The apparatus of claim 15, wherein the measuring unit comprises:
an activity measuring unit for measuring each activity value of a first cyclin-dependent kinase (first CDK) and a second cyclin-dependent kinase (second CDK) from the malignant tumor; and
an expression level measuring unit for measuring each expression level of the first CDK and the second CDK from the malignant tumor;
wherein the measurement value obtainer obtains a first specific activity of the first CDK based on the measurement results of the activity measuring unit and the expression level measuring unit, a second specific activity of the second CDK based on the measurement results of the activity measuring unit and the expression level measuring unit, and a specific activity ratio of the first specific activity and the second specific activity;
wherein preparer for preparing the diagnosis support information of cancer by comparing the obtained specific activity ratio and the reference value stored in the memory.

17. An apparatus for supporting diagnosis of cancer, comprising:
a memory means for storing a predetermined reference value;
a measurement value acquiring means for acquiring a measurement value of a predetermined item from a malignant tumor collected from a cancer patient;
a diagnosis support information preparing means for preparing the diagnosis support information of cancer by comparing the acquired measurement value and the reference value; and
a change accepting means for accepting change of the reference value and for storing the changed reference value in the memory means.

18. The apparatus of claim 17, wherein the diagnosis support information preparing means prepares the diagnosis support information by comparing the acquired measurement value and the changed reference value after the changed reference value has been stored in the memory means.

19. The apparatus of claim 17, comprising a display means and a display controlling means for displaying the obtained diagnosis support information on the display means.

20. The apparatus of claim 19, wherein the display controlling means displays a reference value changing screen for inputting the change of the reference value on the display means, and the change accepting means accepts the input change of the reference value.
